(19) United States
(12) Patent Application Publication
(10) Pub. No.: US 2022/0401402 A1
(43) Pub. Date: Dec. 22, 2022

(54) COMPOUNDS FOR PREVENTING MIGRATION OF CANCER CELLS

(71) Applicant: Deutsches Krebsforschungszentrum, Heidelberg (DE)

(72) Inventors: Joao SECO, Heidelberg (DE); Joana Filipa GUERREIRO, Heidelberg (DE); Marco GOMES, Heidelberg (DE); Adolfo HORN, Florianópolis SC (BR); Christiane Femandes HORN, Florianópolis SC (BR); Francesca PAGLIARI, Heidelberg (DE)

(73) Assignee: Deutsches Krebsforschungszentrum, Heidelberg (DE)

(21) Appl. No.: 17/766,128

(22) PCT Filed: Oct. 2, 2020
(86) PCT No.: PCT/EP2020/077661

§ 371 Date: Apr. 1, 2022

(30) Foreign Application Priority Data
Oct. 4, 2019 (EP) 19201557.6
Feb. 24, 2020 (EP) 20159037.9
Mar. 6, 2020 (EP) 20161483.1

(51) Int. Cl. A61K 31/295; A61K 31/30; A61K 31/315; A61P 35/00

(52) U.S. Cl. CPC A61K 31/295; A61K 31/30; A61K 31/315; A61P 35/00

(57) ABSTRACT

The present invention relates to a composition for use in preventing migration of cancer cells in a subject known or suspected to suffer from cancer, the composition comprising at least one metal complex having the structure (I), formula (I), wherein M is a metal, preferably selected from the group consisting of copper, iron, manganese and zinc, X is X^{a} or X^{b}, wherein X^{a} is selected from the group consisting of O, S and —N(R¹)—, wherein R¹ is H or alkyl, and wherein X^{b} is a group forming a coordinate covalent bond to a second metal M′, preferably a group O, S or —N(R¹)—, wherein M′ is preferably selected from the group consisting of copper, iron, manganese and zinc, and wherein M′ and M may be the same or different and are preferably the same, Z¹ and Z² are independently of each other a, substituted or unsubstituted, -Aryl-O—, -Aryl-N— or heteroaryl group, Y is Y^{a} or Y^{b}, wherein Y^{a} is selected from the group consisting of H, alkyl, —OH, —SH, halogen, and —NR³R⁴, wherein R³ and R⁴, are independently of each other selected from H and alkyl, preferably R³ and R⁴ are both H, and wherein Y^{b} is a group forming a coordinate covalent bond to M or M′, preferably Y^{b} is a group O, S or —N(R^{Yb1}R^{Yb2}), wherein R^{Yb1} and R^{Yb1}, are, independently of each other, H or alkyl, preferably H, and wherein n and m are integers, which are independently of each other, 0 or 1, Y² is water or a halogen, and wherein Y³ is water or a halogen. The present invention further relates to a combined preparation comprising the aforesaid composition as well as to an in vitro method, for determining whether cancer cells are susceptible to immobilizing by the aforesaid compound.

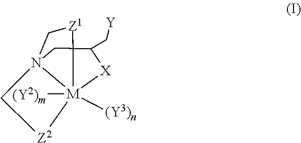

The present invention relates to a composition for use in preventing migration of cancer cells in a subject known or suspected to suffer from cancer, the composition comprising at least one metal complex having the structure (I)

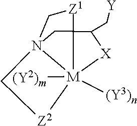

wherein

M is a metal, preferably selected from the group consisting of copper, iron, manganese and zinc, X is X^{a} or X^{b}, wherein X^{a} is selected from the group consisting of O, S and —N(R¹)—, wherein R¹ is H or alkyl, and wherein X^{b} is a group forming a coordinate covalent bond to a second metal M′, preferably a group O, S or —N(R¹)—, wherein M′ is preferably selected from the group consisting of copper, iron, manganese and zinc, and wherein M′ and M may be the same or different and are preferably the same, Z¹ and Z², are independently of each other a, substituted or unsubstituted, -Aryl-O—, -Aryl-N— or heteroaryl group, Y is Y^{a} or Y^{b}, wherein Y^{a} is selected from the group consisting of H, alkyl, —OH, —SH, halogen, and —NR³R⁴, wherein R³ and R⁴, are independently of each other selected from H and alkyl, preferably R3 and R4 are both H, and wherein Y^{b} is a group forming a coordinate covalent bond to M or M′, preferably Y^{b} is a group O, S or —N(R^{Yb1}R^{Yb2}), wherein R^{Yb1} and R^{Yb1}, are, independently of each other, H or alkyl, preferably H, and wherein n and m are integers, which are independently of each other, 0 or 1, Y² is water or a halogen, and wherein Y³ is water or a halogen. The present invention further relates to a combined preparation comprising the aforesaid composition as well as to an in vitro method for determining whether cancer cells are susceptible to immobilizing by the aforesaid compound.

The most common primary malignant tumors of the central nervous system in adults are gliomas, which correspond to about 80% of all the malignant brain tumors diagnosed. The treatment of gliomas changes according to the degree of the disease and the patient's condition, but usually includes surgery for maximum resection of the tumor, followed by radiotherapy or chemotherapy. This is made even more difficult by the fact that these tumors are often highly invasive, metastizing into the surrounding tissues and causing recurrence of the disease (Chen et al. (2017), J. Pharmacol. Sci. 134, 59-67).

Research exploring metal-based compounds as chemotherapeutic drugs for the treatment of cancer has increased since the discovery of cisplatin-based chemotherapy. Redox activity, different reactivity in organic substrates, and different coordination modes are some of the characteristics of metal complexes that can be explored in the design of new chemotherapeutic drugs. E.g. binuclear copper complexes were proposed for tumor treatment in BR 10 2014 022630 A2 and in BR 10 2014 017397 A2. In recent years, the number of studies describing metal complexes as metastasis inhibitors has increased, mainly by exploring the modulation of the Epithelial-Mesenchymal Transition (EMT) phenomenon (cf. e.g. He et al. (2017), J. Organomet. Chem. 842, 82-92), Matrix MetalloProteinase (MMP) activity, and Reactive Oxygen species (ROS) production (cf. e.g. Gu et al. (2019), Eur. J. Med. Chem. 164, 654-664) as specific targets.

There is, nonetheless, still a need in the art for improved means and methods for cancer treatment, in particular for preventing cancer cell migration. This problem is solved by the means and methods disclosed herein.

In accordance, the present invention relates to a composition for use in preventing migration of cancer cells in a subject known or suspected to suffer from cancer, the composition comprising at least one metal complex having the structure (I)

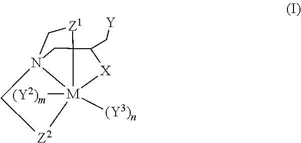

wherein

M is a metal, preferably selected from the group consisting of copper, iron, manganese and zinc, X is X^{a} or X^{b}, wherein X^{a} is selected from the group consisting of O, S and —N(R¹)—, wherein R¹ is H or alkyl, and wherein X^{b} is a group forming a coordinate covalent bond to a second metal M′, preferably a group O, S or —N(R¹)—, wherein M′ is preferably selected from the group consisting of copper, iron, manganese and zinc, and wherein M′ and M may be the same or different and are preferably the same, Z¹ and Z², are independently of each other a, substituted or unsubstituted, -Aryl-O—, -Aryl-N— or heteroaryl group, Y is Y^{a} or Y^{b}, wherein Y^{a} is selected from the group consisting of H, alkyl, —OH, —SH, halogen, and —NR³R⁴, wherein R³ and R⁴, are independently of each other selected from H and alkyl, preferably R3 and R4 are both H, and wherein Y^{b} is a group forming a coordinate covalent bond to M or M′, preferably Y^{b} is a group O, S or —N(R^{Yb1}R^{Yb2}), wherein R^{Yb1} and R^{Yb1} are, independently of each other, H or alkyl, preferably H, and wherein n and m are integers, which are independently of each other, 0 or 1, Y² is water or a halogen, and wherein Y³ is water or a halogen.

Further, the present invention relates to a metal complex, preferably as described above, for use in preventing migration of cancer cells in a subject known or suspected to suffer from cancer, the metal complex being obtained or obtainable by reacting a metal salt comprising a metal ion M* with a ligand having the structure (I*)

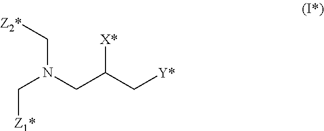

wherein X* is selected from the group consisting of —OH, SH, —N(R¹)(R²)—, wherein R¹ is H or alkyl, and wherein R² is H or alkyl; Z^{1*} and Z^{2*} are independently of each other, a substituted aryl or a, substituted or unsubstituted, heteroaryl group; wherein the substituted aryl group is preferably substituted with an hydroxyl or amine group, Y* is selected from the group consisting of H, alkyl, —OH, —SH, halogen and —NR³R⁴; and wherein M* is selected from the group consisting of copper, iron, and manganese.

Further, the present invention relates to a composition for use in preventing migration of cancer cells in a subject known or suspected to suffer from cancer, the composition comprising at least one copper complex, the complex comprising the ligand

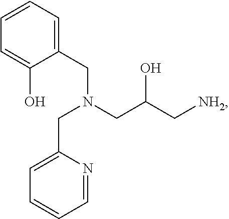 [SMILES: NCC(O)CN(Cc1ccccn1)Cc1ccccc1O]

wherein the complex is preferably obtained upon reaction of CDCl₂ with the ligand and wherein the complex preferably displays a ratio of Cu:ligand:Cl of 2:1:1.

Further, the present invention also relates to a composition for use in preventing migration of cancer cells in a subject known or suspected to suffer from cancer, the composition comprising at least one metal complex, the complex having a structure selected from the group consisting of:

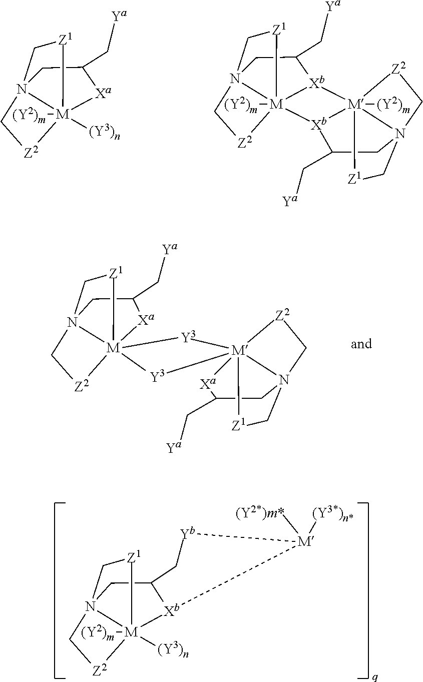

wherein

M is a metal, preferably selected from the group consisting of copper, iron, manganese and zinc, X is X^{a} or X^{b}, wherein X^{a} is selected from the group consisting of O, S and —N(R¹)—, wherein R¹ is H or alkyl, wherein M′ is a further metal and is preferably selected from the group consisting of copper, iron, manganese and zinc, and wherein M′ and M may be the same or different and are preferably the same, Z¹ and Z², are independently of each other a, substituted or unsubstituted, -Aryl-O—, -Aryl-N— or heteroaryl group, Y is Y^{a} or Y^{b}, wherein Y^{a} is selected from the group consisting of H, alkyl, —OH, —SH, halogen, and —NR³R⁴, wherein R³ and R⁴, are independently of each other selected from H and alkyl, preferably R3 and R4 are both H, and wherein Y^{b} is a group forming a coordinate covalent bond to M or M′, preferably Y^{b} is a group O, S or —N(R^{Yb1}R^{Yb2}), wherein R^{Yb1} and R^{Y}bi are, independently of each other, H or alkyl, preferably H, and wherein n and m are integers, which are independently of each other, 0 or 1, Y² is water or a halogen, and wherein Y³ is water or a halogen, wherein n* and m* are integers, which are independently of each other, 0 or 1, Y^{2*} is a solvent molecule or a halogen, preferably water, methanol or a halogen, more preferably, water, methanol or —Cl, and wherein Y^{3*} is a solvent molecule or a halogen, preferably water, methanol or a halogen, more preferably, water, methanol or —Cl, and wherein q is an integer of from 2 to 5, preferably q is 2 or 3, more preferably 2. Preferably, m and n are 0.

Preferably, the present invention also relates to a composition for use in preventing migration of cancer cells in a subject known or suspected to suffer from cancer, the composition comprising at least one metal complex, the complex having the structure

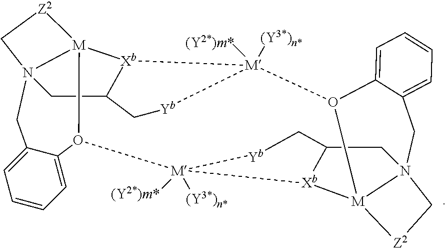

wherein

M, and M′ are metals, which are preferably independently of each other selected from the group consisting of copper, iron, manganese and zinc, wherein M′ and M″ may be the same or different and are preferably the same, wherein X^{a} is selected from the group consisting of O, S and —N(R¹)—, wherein R¹ is H or alkyl, Z¹ and Z², are independently of each other a, substituted or unsubstituted, -Aryl-O—, -Aryl-N— or heteroaryl group, wherein Y^{b} is a group O, S or —N(R^{Yb1}R^{Yb2}), wherein R^{Y}bi and R^{Y}bi are, independently of each other, H or alkyl, preferably H, wherein n* and m* are integers, which are independently of each other, 0 or 1, Y^{2*} is a solvent molecule or a halogen, preferably water, methanol or a halogen, more preferably, water, methanol or —Cl, and wherein Y^{3*} is a solvent molecule or a halogen, preferably water, methanol or a halogen, more preferably, water, methanol or —Cl, and wherein preferably M, and M′ are copper.

Further, the present invention also relates to a complex as such having the structure:

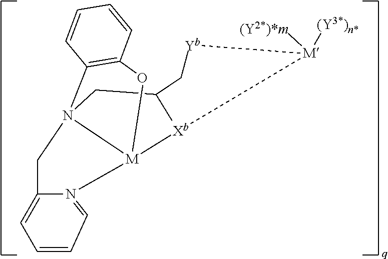

wherein with M and M′ are copper, Y^{b} is NH2, X^{b} is a group forming a coordinate covalent bond to a second metal M′, preferably a group O, S or —N(R¹)—, more preferably O, and wherein m* and n* are both 1, wherein Y^{2*} is a solvent molecule or a halogen, preferably water, methanol or a halogen, more preferably, water, methanol or —Cl, and wherein Y^{3*} is a solvent molecule or a halogen, preferably water, methanol or a halogen, more preferably, water, methanol or —Cl, in particular wherein Y^{2*} and Y3* are both —Cl, and wherein q is an integer in the range of from 2 to 5, preferably 2 or 3, more preferably 2.

Further, the present invention relates to a complex for use as a medicament, preferably for use in preventing or treating cancer, more preferably for use in preventing migration of cancer cells in a subject known or suspected to suffer from cancer, the complex having the structure:

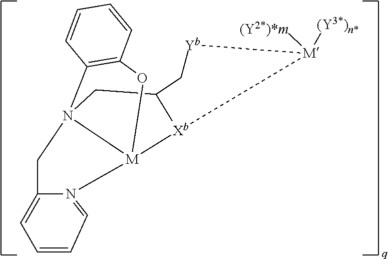

wherein with M and M′ are copper, Y^{b} is NH₂, X^{b} is a group forming a coordinate covalent bond to a second metal M′, preferably a group O, S or —N(R¹)—, more preferably O, and wherein m* and n* are both 1, wherein Y^{2*} is a solvent molecule or a halogen, preferably water, methanol or a halogen, more preferably, water, methanol or —Cl, and wherein Y^{3*} is a solvent molecule or a halogen, preferably water, methanol or a halogen, more preferably, water, methanol or —Cl, in particular wherein Y^{2*} and Y3* are both —Cl, and wherein q is an integer in the range of from 2 to 5, preferably 2 or 3, more preferably 2.

Further, the present invention also relates to a copper complex, the complex comprising the ligand

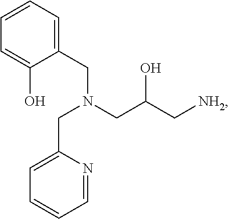 [SMILES: NCC(O)CN(Cc1ccccn1)Cc1ccccc1O]

wherein the complex is preferably obtained or obtainable upon reaction of CuCl₂, with the ligand and wherein the complex preferably displays a ratio of Cu:ligand:Cl of 2:1:1, and wherein the complex more preferably has the structure:

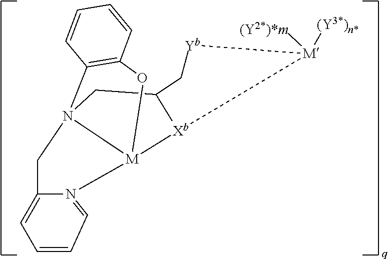

wherein with M and M′ are copper, Y^{b} is NH₂, X^{b} O, and wherein m* and n* are both 1, wherein Y^{2*} and Y^{3*} are, independently of each other selected from the group consisting of a solvent molecule, water and —Cl, more preferably both, Y^{2*} and Y^{3*} are preferably Cl, and wherein q is an integer in the range of from 2 to 5, preferably 2 or 3, more preferably 2.

Further, the present invention also relates to a copper complex for use as a medicament, preferably for use in preventing or treating cancer, more preferably for use in preventing migration of cancer cells in a subject known or suspected to suffer from cancer, the complex comprising the ligand

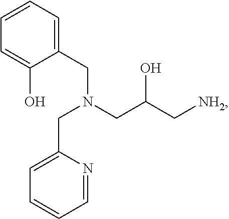 [SMILES: NCC(O)CN(Cc1ccccn1)Cc1ccccc1O]

wherein the complex is preferably obtained or obtainable upon reaction of CuCl₂, with the ligand and wherein the complex preferably displays a ratio of Cu:ligand:Cl of 2:1:1, and wherein the complex more preferably has the structure:

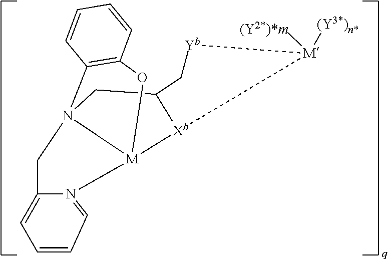

wherein with M and M′ are copper, Y^{b} is NH₂, X^{b} O, and wherein m* and n* are both 1, wherein Y^{2*} and Y^{3*} are, independently of each other selected from the group consisting of a solvent molecule, water and —Cl, more preferably both, Y^{2*} and Y^{3*} are preferably Cl, and wherein q is an integer in the range of from 2 to 5, preferably 2 or 3, more preferably 2.

Further, the present invention also relates to a compound having the structure:

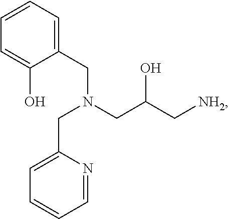 [SMILES: NCC(O)CN(Cc1ccccn1)Cc1ccccc1O]

as such.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, as used in the following, the terms “preferably”, “more preferably”, “most preferably”, “particularly”, “more particularly”, “specifically”, “more specifically” or similar terms are used in conjunction with optional features, without restricting further possibilities. Thus, features introduced by these terms are optional features and are not intended to restrict the scope of the claims in any way. The invention may, as the skilled person will recognize, be performed by using alternative features. Similarly, features introduced by “in an embodiment” or similar expressions are intended to be optional features, without any restriction regarding further embodiments of the invention, without any restrictions regarding the scope of the invention and without any restriction regarding the possibility of combining the features introduced in such way with other optional or non-optional features of the invention.

As used herein, the term “standard conditions”, if not otherwise noted, relates to IUPAC standard ambient temperature and pressure (SATP) conditions, i.e. preferably, a temperature of 25° C. and an absolute pressure of 100 kPa; also preferably, standard conditions include a pH of 7. Moreover, if not otherwise indicated, the term “about” relates to the indicated value with the commonly accepted technical precision in the relevant field, preferably relates to the indicated value ±20%, more preferably +10%, most preferably +5%. Further, the term “essentially” indicates that deviations having influence on the indicated result or use are absent, i.e. potential deviations do not cause the indicated result to deviate by more than +20%, more preferably ±10%, most preferably +5%. Thus, “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Preferably, a composition consisting essentially of a set of components will comprise less than 5% by weight, more preferably less than 3% by weight, even more preferably less than 1%, most preferably less than 0.1% by weight of non-specified component(s).

### The Metals M and M′

As described above, the metals M and M′ are, preferably, independently of each other, selected from the group consisting of iron, copper, manganese and zinc. If both metals are present in the complex, i.e. if the complex is e.g. a binuclear complex, the metals may be the same or may be different. Preferably, M and M′ are the same as M′ and M.

More preferably, M and M′, are, independently of each other selected from the group consisting of Fe(III), Cu(II), Mn(II) and Zn(II), with Fe(III) and Cu(II) being particularly preferred. Most preferably, M and M′ are Cu(II).

Thus, the present invention also relates to a composition as described above, and to a composition comprising a metal complex obtained or obtainable as described above, as well as to a complex as described above, and to a complex obtained or obtainable as described above, wherein the metal M is copper or iron, preferably Fe(III) or Cu(II) and if present, M′ is copper or iron, preferably Fe(III) or Cu(II), and wherein most preferably M and M′ are the same and in particular Cu(II).

According to a preferred embodiment, the present invention also relates to a composition as described above, and to a composition comprising a metal complex obtained or obtainable as described above, as well as to a complex as such, as described above, and a complex obtained or obtainable as described above, wherein the metal complex is multinuclear, preferably a tetranuclear complex, comprising the metals M and M′, that is twice the metal M and twice the metal M′, wherein M is copper or iron, preferably Fe(III) or Cu(II) and M′ is copper or iron, preferably Fe(III) or Cu(II), wherein most preferably M and M′ are the same, and in particular all Cu(II).

M* The metals salt consequently, preferably, comprises a metal ion M* of iron, copper, manganese or zinc, more preferably of copper or iron. As salts, any salts suitable to be reacted with ligand L* may be used. Preferably, the metal salts are selected from the group consisting of Fe(III), Cu(II), Mn(II) and Zn(II) salts, with Fe(III) and Cu(II) salts being particularly preferred.

As preferred salts, the following salts shall be mentioned: CuCl₂.H₂O, Cu(ClO₄)₂.6H₂O, FeCl₃.6H₂O, Fe(ClO₄)₃.xH₂O, MnCl₂.4H₂O, Mn(ClO₄)₂.xH₂O, ZnCl₂, and Zn(ClO₄)₂.6H₂O.

### The Ligand (I*)

As described above, ligand has the structure (I*), wherein X* is selected from the group consisting of —OH, SH, —N(R¹)(R²)—, wherein R¹ is H or alkyl, and wherein R² is H or alkyl; Z¹* and Z^{2*} are independently of each other, a substituted aryl or a, substituted or unsubstituted, heteroaryl group; wherein the substituted aryl group is preferably substituted with an hydroxyl or amine group, Y* is selected from the group consisting of H, alkyl, —OH, —SH, halogen and —NR³R⁴.

The term aryl as used within the context of the present invention, refers to 5- and 6-membered aromatic rings, and polycyclic aromatic groups (aryl groups), for example tricyclic or bicyclic aryl groups. Polycyclic aromatic groups can also contain non-aromatic rings. As mentioned above, the aryl is preferably substituted. The term “substituted” in this context means that the aryl group contains preferably at least one substituent, such as one, two, three or four substituents. Preferably, the aryl group comprises at least one substituent which may function as a donor group, i.e. which comprises at least one free electron pair, i.e. acts as a Lewis bases, or an ionic group capable of forming a coordinate covalent bond to a metal. As suitable donor groups, e.g. hydroxyl, halogen, —SH and amine groups shall be mentioned. Preferably, the aryl group comprises at least one amine or hydroxyl group, more preferably at least one hydroxyl group. It is to be understood that the aryl group may comprise in addition further substituents, such as substituents selected from the group consisting of hydroxyl, halogen, —SH, amine groups, alkyl and solubility enhancing groups. The term solubility enhancing groups refers to such substituents which enhance the solubility of the compound under physiological conditions, such as carboxy groups, PEG groups and the like.

The term “heteroaryl”, as used in the context of the present invention, refer to 5- and 6-membered aromatic rings, and polycyclic aromatic groups, for example tricyclic or bicyclic aryl groups, containing one or more, for example 1 to 4, such as 1, 2, 3, or 4, heteroatoms in the ring system. If more than one heteroatom is present in the ring system, the at least two heteroatoms that are present can be identical or different. Suitable heteroaryl groups are known to the skilled person. The following heteroaryl residues may be mentioned, as non-limiting examples: benzodioxolyl, pyrrolyl, furanyl, thiophenyl, thiazolyl, isothiaozolyl, imidazolyl, triazolyl, tetrazolyl, pyrazolyl, oxazolyl, isoxazolyl, pyridinyl, pyrazinyl, pyridazinyl, benzoxazolyl, benzodioxazolyl, benzothiazolyl, benzoimidazolyl, benzothiophenyl, methylenedioxyphenylyl, napthridinyl, quinolinyl, isoqunilyinyl, indolyl, benzofuranyl, purinyl, benzofuranyl, deazapurinyl, pyridazinyl and indolizinyl.

It is to be understood that upon reaction of the ligand (I*) with the metal M, and optionally M′, the complex is formed. Thus, X* is reacted to X, Y* is reacted to Y, Z₁* is reacted to Z₁ and Z₂^{*} is reacted to Z₂.

### Residue Z₁*

Z₁* is a substituted aryl or a, substituted or unsubstituted, heteroaryl group, wherein the substituted aryl group is preferably substituted with a hydroxyl or amine group.

Preferably, Z₁* is a substituted phenyl group, preferably a phenyl group substituted at least with a hydroxyl group, more preferably a phenyl group substituted in ortho-position with a hydroxyl group and having the following structure:

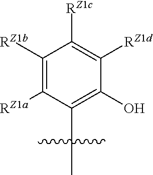

with R^{Z1a}, R^{Z1b}, R^{Z1c} and R^{Z1d}, being, independently of each other, selected from the group consisting of H, optionally substituted, alkyl, and, optionally substituted, aryl. Preferably, R^{Z1a}, R^{Z1b}, R^{Z1c} and R^{Z1d} are H.

### Residue Z₂*

Z₂* is a substituted aryl or a, substituted or unsubstituted, heteroaryl group, wherein the substituted aryl group is preferably substituted with a hydroxyl or amine group. More preferably, Z₂* is a, substituted or unsubstituted, pyridyl group, more preferably a group having the structure

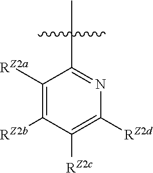

with R^{Z2a}, R^{Z2b}, R^{Z2c} and R^{Z2d} being, independently of each other, selected from the group consisting of H, optionally substituted, alkyl, and, optionally substituted, aryl. Preferably, R^{Z2a}R^{Z2b}, R^{Z2c} and R^{Z2d} are H.

### Residue X*

X* is selected from the group consisting of —OH, SH, —N(R¹)(R²)—, wherein R¹ is H or alkyl, and wherein R² is H or alkyl; Z^{1*} and Z^{2*}, are independently of each other, a substituted aryl or a, substituted or unsubstituted, heteroaryl group; wherein the substituted aryl group is preferably substituted with an hydroxyl or amine group,

### Residue Y*

Y* is selected from the group consisting of H, alkyl, —OH, —SH, halogen and —NR³R⁴. Preferably, Y* is selected from the group consisting of —Cl, —NH₂, —CH₃ and H, more preferably Y* is —CH₃ and —Cl, more preferably —Cl.

According to a further preferred embodiment, Y* is NH₂.

Preferred ligands (I*) according to the invention are, by way of example:

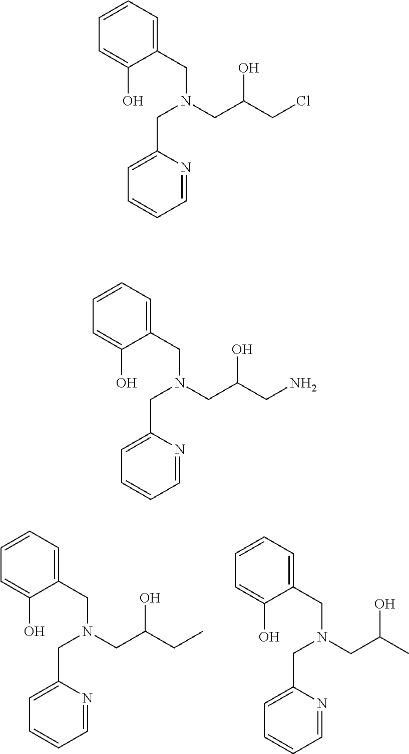 [SMILES: Oc1ccccc1CN(Cc1ccccn1)CC(O)CCl ; NCC(O)CN(Cc1ccccn1)Cc1ccccc1O ; CCC(O)CN(Cc1ccccn1)Cc1ccccc1O ; CC(O)CN(Cc1ccccn1)Cc1ccccc1O]

Preferably, the ligand is

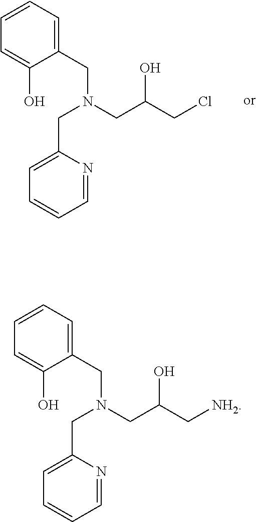 [SMILES: Oc1ccccc1CN(Cc1ccccn1)CC(O)CCl ; NCC(O)CN(Cc1ccccn1)Cc1ccccc1O]

According to a preferred embodiment, the ligand is

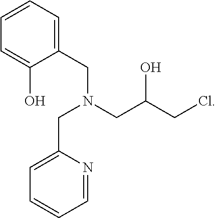 [SMILES: Oc1ccccc1CN(Cc1ccccn1)CC(O)CCl]

More preferably the ligand is

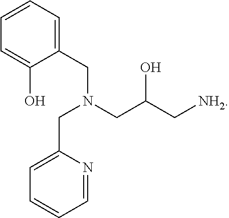 [SMILES: NCC(O)CN(Cc1ccccn1)Cc1ccccc1O]

Advantageously, it was found that complexes, in particular copper complexes, comprising this ligand are water soluble.

Further, the present invention relates to a metal complex, as described above, as well as to a metal complex for use as a medicament, preferably for use in preventing migration of cancer cells in a subject known or suspected to suffer from cancer, as described above, the metal complex being obtained or obtainable by reacting a metal salt comprising a metal ion M* with a ligand having the structure

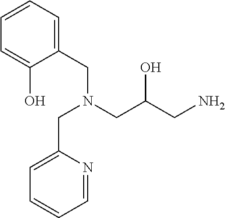

wherein M* is selected from the group consisting of copper, iron, and manganese, more preferably wherein M* is copper, most preferably the metal salt is CuCl₂. Preferably, upon reaction with CuCl₂ and the ligand

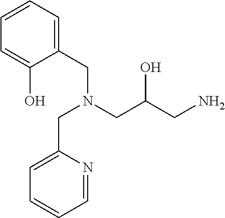 [SMILES: NCC(O)CN(Cc1ccccn1)Cc1ccccc1O]

a complex having the structure

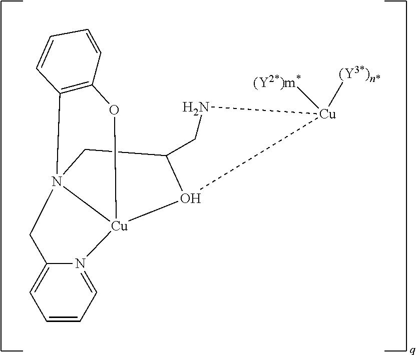

is formed, wherein m* and n* are 1, Y^{2*} is a solvent molecule or a halogen, preferably water, methanol or a halogen, more preferably, water, methanol or —Cl, in particular —Cl, and Y^{3*} is a solvent molecule or a halogen, preferably water, methanol or a halogen, more preferably, water, methanol or —Cl, in particular —Cl, and wherein q is an integer of from 2 to 5, preferably q is 2 or 3, more preferably 2.

Preferably, the metal complex is obtained or obtainable by reacting a CuCl₂ and the ligand in an organic solvent, preferably in an organic solvent comprising an alcohol, more preferably in isopropanol. Preferably, the reaction is carried out at elevated temperatures, such as a temperature in the range of from 40° C. to 100° C., preferably at reflux. Subsequently, the complex is preferably precipitated.

Further, the present invention also relates to a composition comprising a metal complex, as described above, as well as to a metal complex as such, as described above, as well as to a metal complex for use as a medicament, preferably for use in preventing migration of cancer cells in a subject known or suspected to suffer from cancer, as described above, the metal complex being obtained or obtainable by a process comprising
(i) reacting a metal salt comprising a metal ion M* with a ligand (I*), as described above, preferably a ligand having the structure

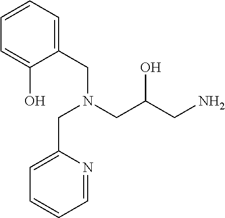 [SMILES: NCC(O)CN(Cc1ccccn1)Cc1ccccc1O]

preferably a copper salt, more preferably CuCl₂, more preferably in an organic solvent
(ii) precipitating the complex and isolating the complex. wherein (i) is preferably carried at elevated temperature, preferably under reflux.

More preferably, in (i) an equimolar amount of the ligand and the copper salt is used. Optionally, the metal complex is further purified using methods known to those skilled in the art.

### The Complex

The complex or the complex of the composition of the invention, or the complex obtained or obtainable as described above, is preferably a mononuclear or binuclear complex or a mixture thereof.

According to an alternative preferred embodiment, the complex is a tetranuclear or multinuclear complex, preferably a tetranuclear complex.

### The Group X

As described above, X is X^{a} or X^{b}, wherein X^{a} is selected from the group consisting of O, S and —N(R¹)—, wherein R¹ is H or alkyl, and wherein X^{b} is a group forming a coordinate covalent bond to a second metal M′, preferably a group O, S or —N(R¹)—.

Thus, according to a first preferred embodiment, X is X^{a}, wherein X^{a} is selected from the group consisting of O, S and —N(R¹)—. More preferably, X^{a} is selected from the group consisting of O, S and —NH—, more preferably O.

According to a second preferred embodiment, X is X^{b}, i.e. a group forming a coordinate covalent bond to a second metal M′, preferably X^{b} is a group O, S or —N(R¹)—, in particular a group O, S or —NH—, more preferably O. In case X is X^{b}, the complex is a binuclear complex comprising metal M and metal M′.

### The Group Y

As described above, Y is Y^{a} or Y^{b}, wherein Y^{a} is selected from the group consisting of H, alkyl, —OH, —SH, halogen, and —NR³R⁴, wherein R³ and R⁴, are independently of each other selected from H and alkyl, preferably R³ and R⁴ are both H, and wherein Y^{b} is a group forming a coordinate covalent bond to M or M′, preferably Y^{b} is a group O, S or —N(R^{Yb1}R^{Yb2}), wherein R^{Yb1} and R^{Yb1}, are, independently of each other, H or alkyl, preferably H.

Thus, according to a first preferred embodiment, Y is Y^{a}, with Y^{a} being selected from the group consisting of H, alkyl, —OH, —SH, halogen, and —NR³R⁴, wherein R³ and R⁴, are independently of each other selected from H and alkyl, preferably R³ and R⁴ are both H. More preferably, Y^{a} is selected from the group consisting of —Cl and —NH₂, more preferably —Cl. According to further particularly preferred embodiment Y^{a} is —NH₂.

According to this preferred embodiment, the complex, in particular has one of the following structures:

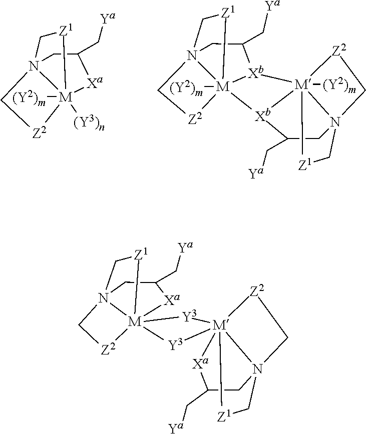

According to a second preferred embodiment, Y is Y^{b}, wherein Y^{b} is a group forming a coordinate covalent bond to M or M′, preferably Y^{b} is a group O or —N(R^{Yb1}R^{Yb2}), wherein R^{Yb1} and R^{Yb1} are, independently of each other, H or alkyl, preferably H, more preferably Y^{b} is —NH₂.

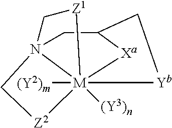

Alternatively, the complex in this case has the structure

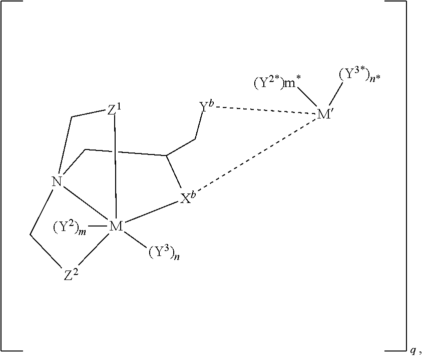

preferably

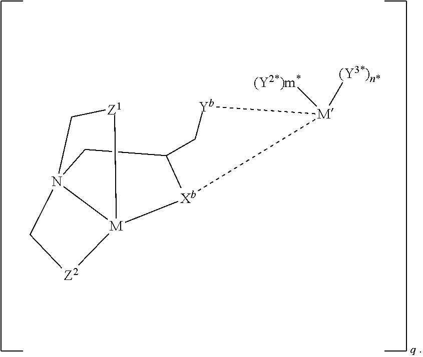

In this case, the complex is e.g. a tetranuclear complex, if q is 2.

According to a particularly preferred embodiment, the complex has the structure

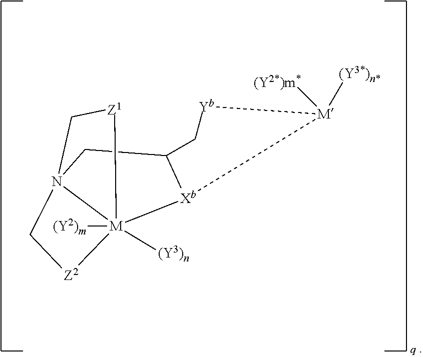

with m and n preferably being 0 and with q preferably being 2.

Typically, the complex thus has one of the following structures:

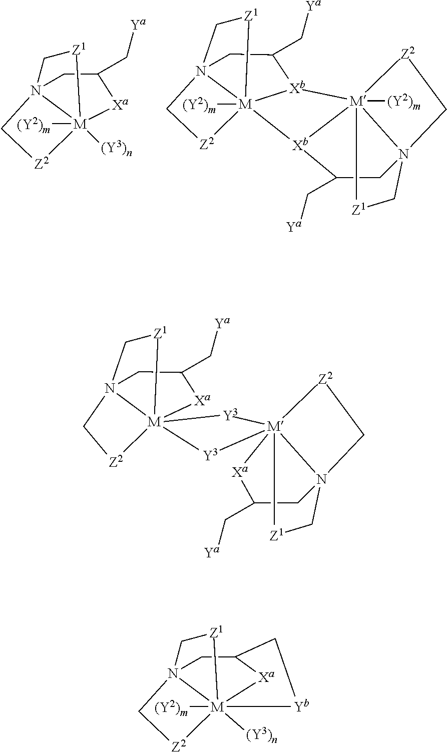

or one of the following structures: PGP-50.Ci

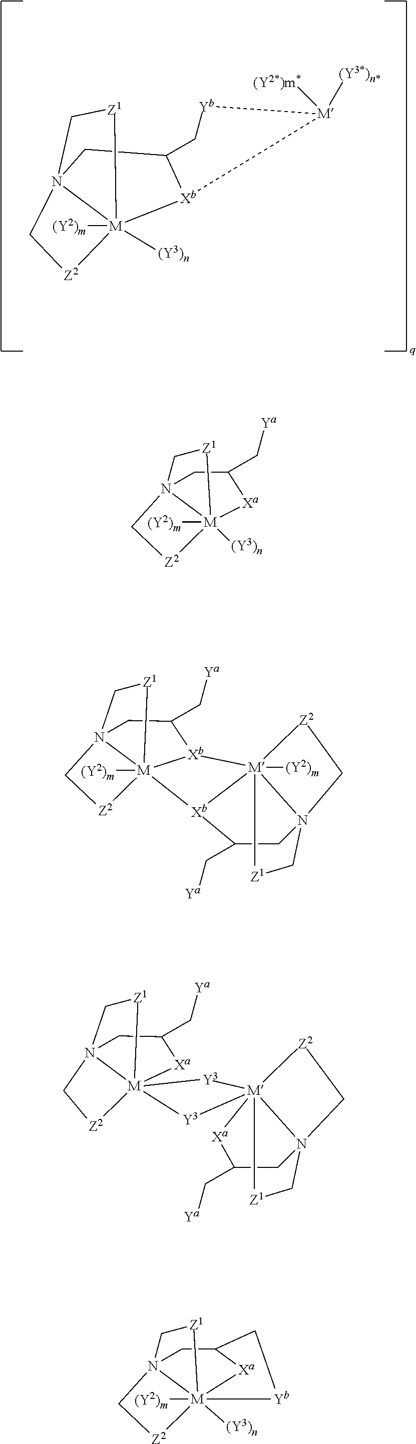

Preferably the complex has the structure:

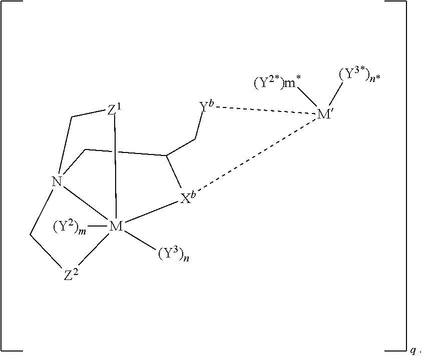

with m and n preferably being 0.

According to a further preferred embodiment, the complex, is selected from the group consisting of the following structures.

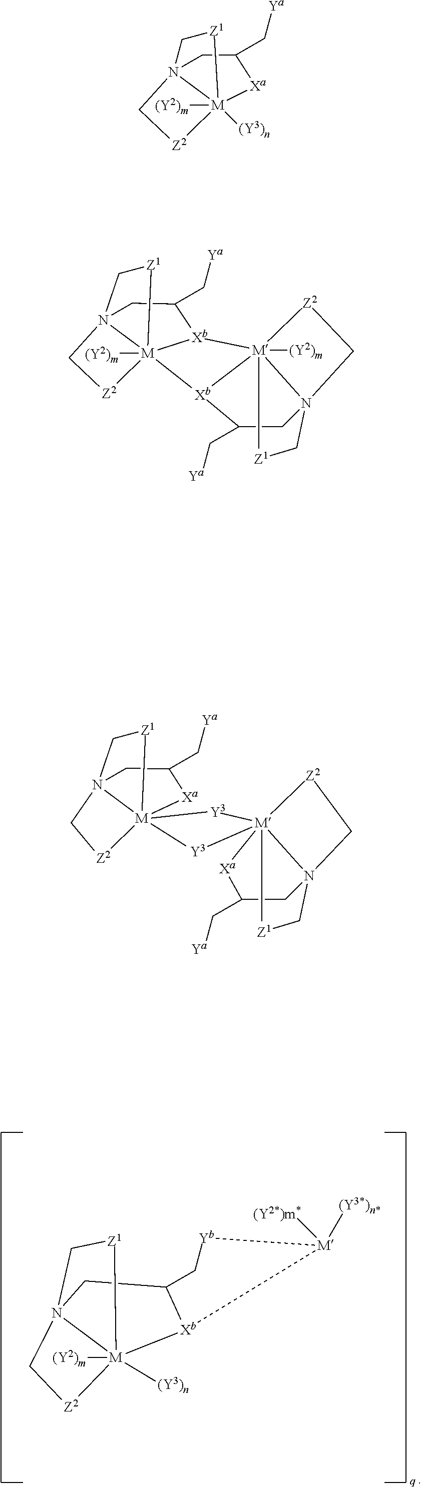

The group Z¹

As described above, Z¹ is preferably, a substituted or unsubstituted, -Aryl-O—, -Aryl-N— or heteroaryl group, such as a substituted or unsubstituted -phenyl-O—, -phenyl-N— or pyridyl group. More preferably Z¹ is a substituted or unsubstituted, -Aryl-O—, preferably a -phenyl-O-group, thus a group having the structure

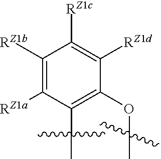

with R^{Z1a}, R^{Z1b}, R^{Z1c} and R^{Z1d}, being, independently of each other, selected from the group consisting of H, optionally substituted, alkyl, and, optionally substituted, aryl. Preferably, R^{Z1a}, R^{Z1b}, R^{Z1c} and R^{Z1d} are H. In this structure the oxygen preferably binds to the metal within the complex. i.e. to M or M′, respectively.

Thus, the complex of the invention, and the complex obtained or obtainable as described above is preferably selected from the group consisting of the following structures:

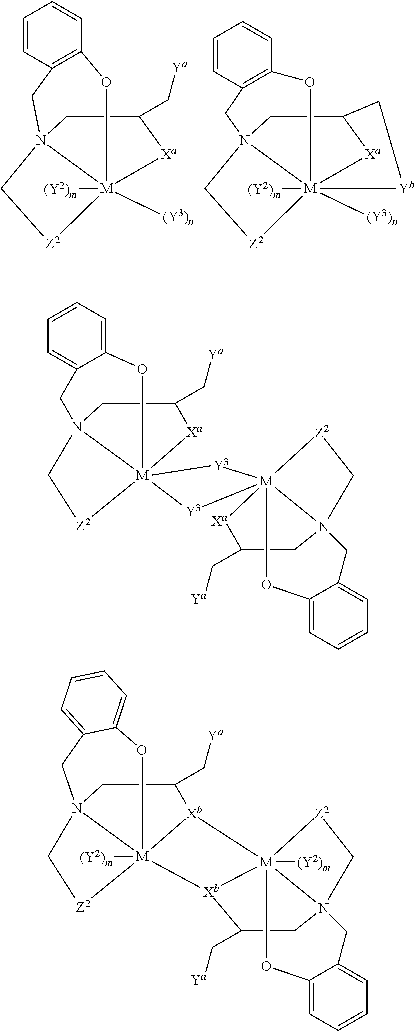

As described above, according to a further preferred embodiment, the complex is a multinuclear complex, such as a tetranuclear complex. In this case, Z¹ is preferably a substituted or unsubstituted, -Aryl-O—, preferably a -phenyl-O— group, thus a group having the structure

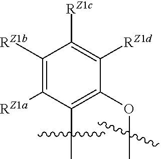

with R^{Z1a}, R^{Z1b}, R^{Z1c} and R^{Z}i1, being, independently of each other, selected from the group consisting of H, optionally substituted, alkyl, and, optionally substituted, aryl. Preferably, R^{Z1a}, R^{Z1b}, R^{Z1c} and R^{Z1d} are H. In this structure the oxygen preferably binds to M or M′, respectively.

In this case, the complex has preferably the structure

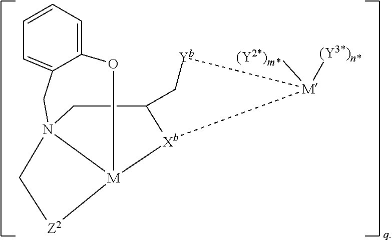

with m and n preferably being 0.

Advantageously, it was found that such complexes, in particular complexes with Y^{b}═NH2 and copper as metal, are water soluble.

According to a preferred embodiment, according to which q is 2, the complex has the structure

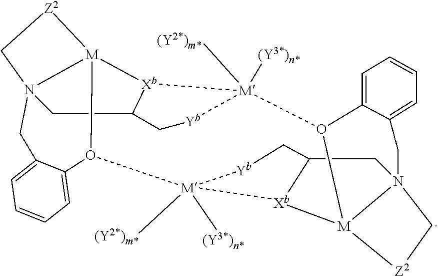

### The Group Z²

As described above, Z² is preferably, a substituted or unsubstituted, -Aryl-O—, -Aryl-N— or heteroaryl group, such as a substituted or unsubstituted -phenyl-O—, -phenyl-N— or pyridyl group. More preferably Z² is a substituted or unsubstituted pyridyl group, thus a group having the structure:

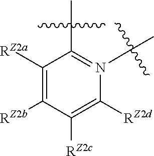

with R^{Z2a}, R^{Z2b}, R^{Z2c} and R^{Z2d} being, independently of each other, selected from the group consisting of H, optionally substituted, alkyl, and, optionally substituted, aryl. Preferably, R^{Z2a}R^{Z2b}, R^{Z2c} and R^{Z2d} are H.

Preferably, the complex of the invention, and the complex obtained or obtainable as described above is thus selected from the group consisting of the following structures:

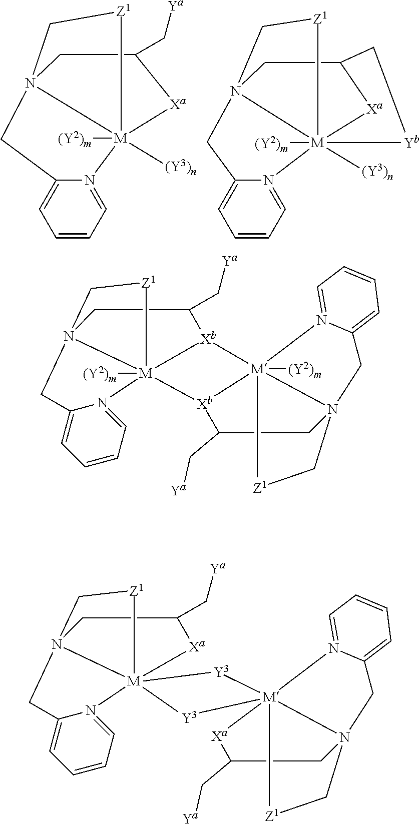

More preferably, the complex of the invention, and the complex obtained or obtainable as described above is preferably selected from the group consisting of the following structures:

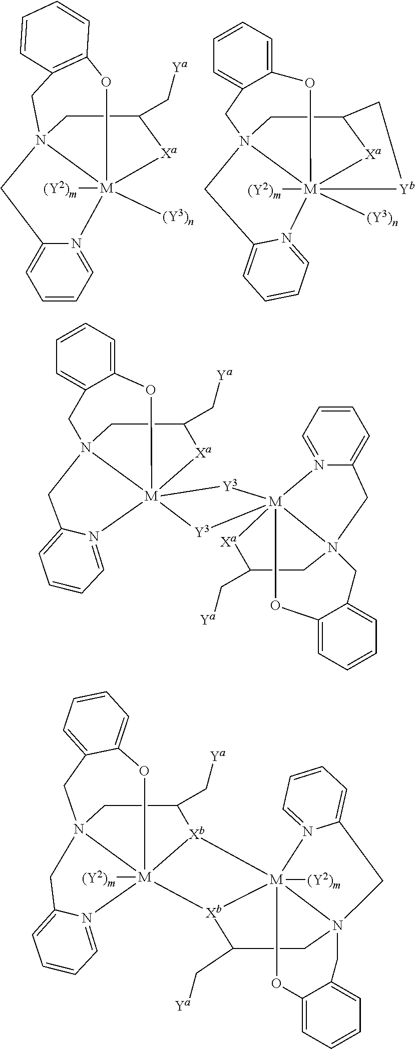

According to a further preferred embodiment, the complex has the structure:

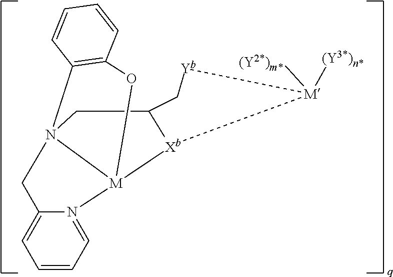

According to a further preferred embodiment, in this complex q is 2, the complex thus having the structure:

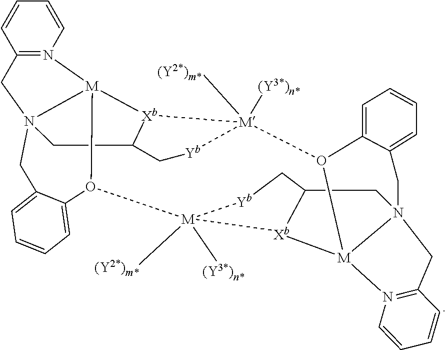

wherein preferably M is the same as M′, and preferably both are copper.

The groups Y2 and Y3, integers m and n

As described above, n and m are integers, which are independently of each other, 0 or 1, Y² is water or a halogen, and Y³ is water or a halogen. Whether or not Y2 and/or Y3 depends on the coordination number of the metal used.

Thus, if e.g copper is used, the complex has preferably the following structure,

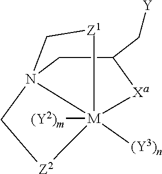

with m being preferably 0 and n being preferably 1. In this case, Y³ is most preferably Cl. Y is preferably Y^{a}. It is to be understood that the complex may be charged such as positively charged.

Thus, the complex preferably has the structure

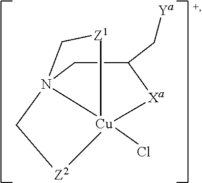

more preferably the structure

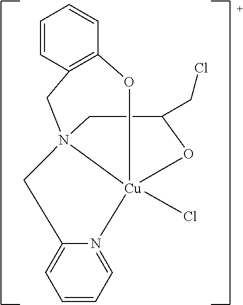

Alternatively, the complex may have the following structure or may be present in a mixture with a complex having the following structure

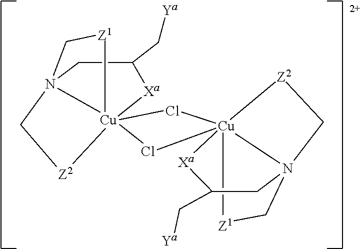

more preferably the following structure:

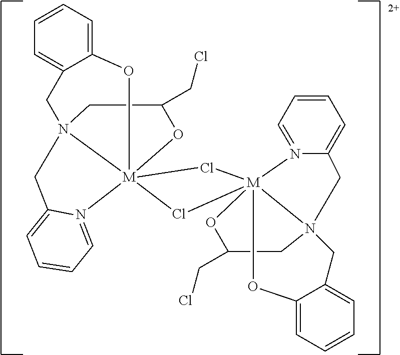

According to an alternative preferred embodiment, the metal is copper, and the complex has the structure

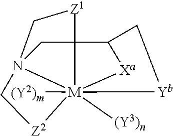

wherein m and n are preferably 0. Preferably, in this case, Y^{b} is —NH₂. Thus, the complex preferably has the structure:

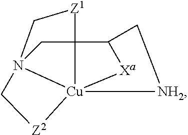

more preferably the structure

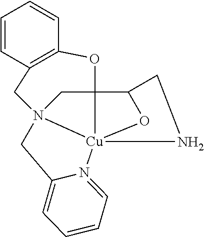

According to a further preferred embodiment, the metal is Fe, and the complex has the structure

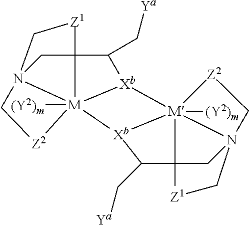

with m being 1 and Y2 being water. Thus. the complex preferably has the structure:

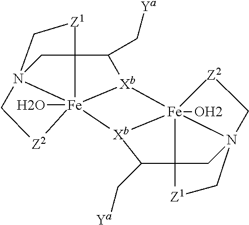

More preferably, the following structure:

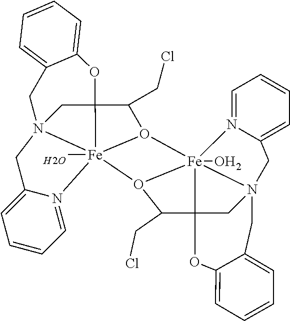

Thus, particularly preferred complexes are selected from the group consisting of:

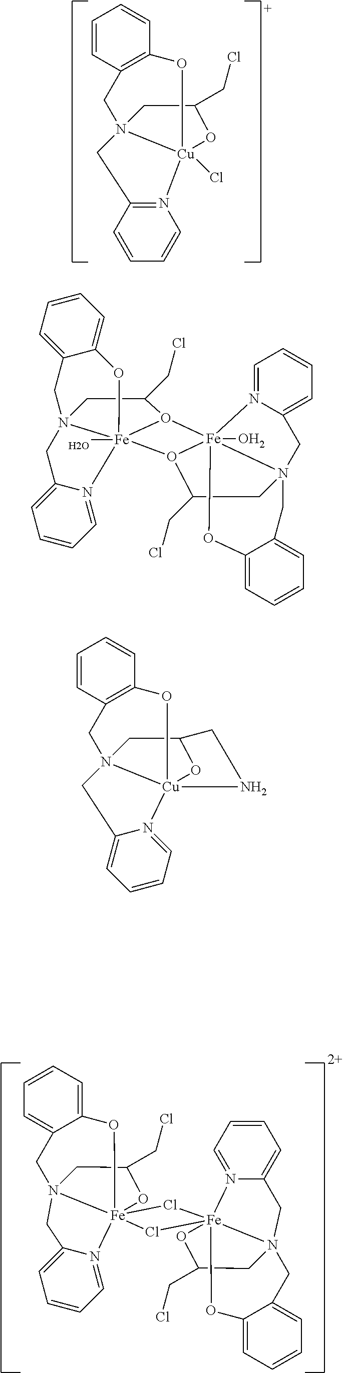

and mixtures of two or more thereof.

As described above, according to a further preferred embodiment, the complex has the structure

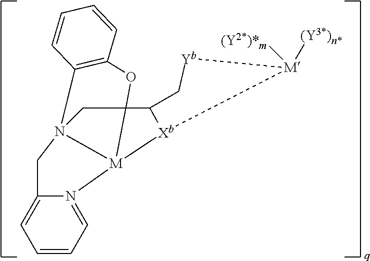

with M and M′ preferably being copper, Y^{b} preferably being NH₂, X^{b}, preferably being O. More preferably q is 0 and the complex is a tetranuclear complex, the complex preferably having the structure:

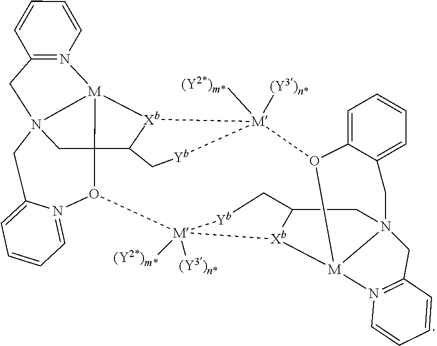

In this case, M is preferably the same as M′, wherein the metals are preferably all Cu, the compound thus preferably having the following structure:

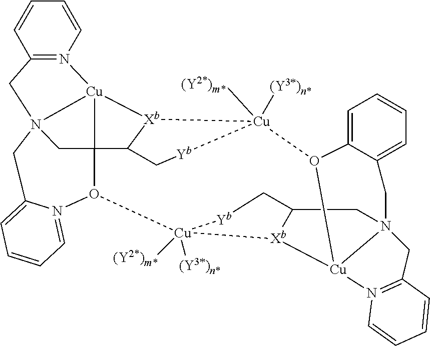

more preferably, the following structure,

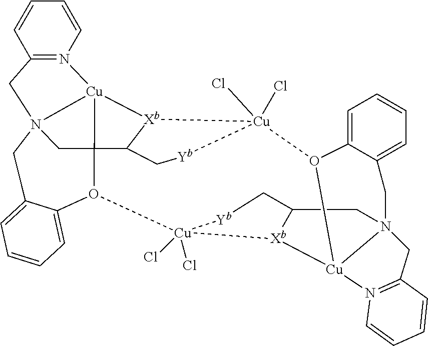

even more preferably, the following structure

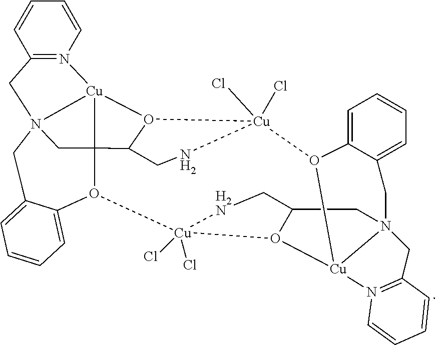

### Synthesis of the Complex of the Invention:

The complex of the present invention is prepared according to methods known to the skilled person, such as described in Horn, A. et al. Synthesis, crystal structure and properties of dinuclear iron(III) complexes containing terminally coordinated phenolate H2O/OH-groups as models for purple acid phosphatases: efficient hydrolytic DNA cleavage. Inorganica Chim. Acta 358, 339-351 (2005); Horn Jr., A. et al. Synthesis, molecular structure and spectroscopic, electrochemical and magnetic properties of a new dinuclear iron complex containing μ-sulfate-di-μ-alkoxo bridges: evaluating the influence of the sulfate bridge on the physicochemical properties of the di-μ-alkoxo-diiron unit. J. Braz. Chem. Soc. 17, 1584-1593 (2006); and Fernandes, C. et al. Synthesis, characterization and antibacterial activity of FeIII, CoII, CuII and ZnII complexes probed by transmission electron microscopy. J. Inorg. Biochem. 104, 1214-1223 (2010).

Preferably, the complex is prepared by
(i) mixing the ligand (I*) and the metals salt M* in a suitable solvent, preferably a solvent selected from the group consisting of methanol, ethanol isopropanol, acetone, acetonitrile, water, dimethylsufoxide, and mixtures of two or more thereof, more preferably a solvent selected from the group consisting of methanol, ethanol, acetonitrile, acetone and mixtures of two or more thereof,
(ii) precipitating the complex from the mixture according to (i).

Preferably, in (i), the mixture is heated, such as to reflux. It is to be understood that further reactants or solvents may be added in (i), such as the addition of water.

Step (i) is preferably carried out by cooling the mixture, preferably to a temperature in the range of from −20° C. to 25° C., more preferably to a temperature in the range of from −10 to +10° C.

The term “composition”, as used herein, relates to a mixture of compounds comprising at least a compound as specified herein and, preferably, at least one carrier. The compounds, preferably the metal complex, comprised in the composition are described herein above. The composition may have any consistency deemed appropriate by the skilled person. Preferably, the composition is a solid composition, e.g. a tablet or a powder, a semisolid composition, e.g. a gel, or, more preferably, a liquid, e.g. a solution or an emulsion.

The composition preferably comprises a carrier. The carrier(s) preferably is/are acceptable in the sense of being compatible with the other ingredients of the composition and being not deleterious to a potential recipient thereof. The carrier(s) preferably is/are selected so as not to affect the biological activity of the composition. Preferably, the composition is sterile, more preferably a sterile solution, most preferably a sterile solution for injection. The carrier is selected by the skilled person such as to achieve the consistency intended and may be, for example, a gel or, preferably, a liquid, more preferably an aqueous liquid. Examples of such carriers are distilled water, physiological saline, Ringer's solutions, dextrose solution, phosphate-buffered saline solution, and Hank's solution. The carrier may include one or more solvents or other ingredients increasing solubility of the compounds comprised in the composition. Further examples of liquid carriers are syrup, oil such as peanut oil and olive oil, water, emulsions, various types of wetting agents, sterile solutions and the like. Suitable carriers comprise those mentioned above and others well known in the art. As is understood by the skilled person, the composition, in particular the pharmaceutical composition, may comprise one or more further compounds; preferably, such additional compounds are selected so as to not affect the biological activity of the composition, in particular of the active compounds, such as the metal complex, and/or is acceptable in the sense of being compatible with the other ingredients of the composition and being not deleterious to a potential recipient thereof.

Preferably, the composition according to the present specification is a pharmaceutical composition; thus, preferably, the carrier is a pharmaceutically acceptable carrier. In addition, the pharmaceutical composition or formulation may also include other carriers, adjuvants, stabilizers and/or other compounds deemed appropriate by the skilled person, e.g. for galenic purposes. As referred to herein, the compound as specified herein above, in particular the metal complex as specified, is the “active compound” of the preparation, although “further active compounds”, which are referred to under this term, may be present. Preferably, the active compound and the further active compound, i.e. preferably the active compounds, are pharmaceutically active compounds. Specific pharmaceutical compositions are prepared in a manner well known in the pharmaceutical art and comprise at least one active compound referred to herein above, preferably in admixture or otherwise associated with at least one pharmaceutically acceptable carrier or diluent. For making those specific pharmaceutical compositions, the active compound(s) will usually be mixed with a carrier or the diluent. The resulting formulations are to be adapted to the mode of administration, i.e. in the forms of tablets, capsules, suppositories, solutions, suspensions or the like. Dosage recommendations shall be indicated in the prescriber's or user's instructions in order to anticipate dose adjustments depending on the considered recipient.

The pharmaceutical composition is, preferably, administered topically or, more preferably, systemically. Suitable routes of administration conventionally used for drug administration are topical, intravenous, or parenteral administration as well as inhalation. Preferably, administration is systemic, more preferably intravenously. However, depending on the nature and mode of action of the specific compound(s) administered and on the clinical situation, the pharmaceutical composition may be administered by other routes as well. In particular, in case the cancer is a brain cancer, intracranial administration may be envisaged. Also, the pharmaceutical composition may be administered topically, e.g. as a tablet, in particular as a time-delay preparation, which is preferably implanted during surgery removing a tumor. Other modes of administration are specified elsewhere herein. Moreover, the pharmaceutical composition can be administered in combination with other further active compounds either in a common pharmaceutical composition or as separated pharmaceutical compositions wherein said separated pharmaceutical compositions may be provided in form of a kit of parts.

The pharmaceutical composition is, preferably, administered in conventional dosage forms prepared by combining the active compound with standard pharmaceutical carriers according to conventional procedures. These procedures may involve mixing or dissolving the ingredients as appropriate to obtain the desired preparation. It will be appreciated that the form and character of the pharmaceutically acceptable carrier or diluent is dictated by the amount of active ingredient with which it is to be combined, the route of administration and other well-known variables. Similarly, the carrier or diluent may include time delay material well known in the art, such as glyceryl mono-stearate or glyceryl distearate alone or with a wax. A therapeutically effective dose refers to an amount of the active compound to be used in a pharmaceutical composition of the present invention which provides the effect referred to in this specification. Therapeutic efficacy and toxicity of such compounds can be determined by standard pharmaceutical procedures in cell cultures or experimental animals, e.g., ED₅₀ (the dose therapeutically effective in 50% of the population) and LD₅₀ (the dose lethal to 50% of the population). The dose ratio between therapeutic and toxic effects is the therapeutic index, and it can be expressed as the ratio, LD₅₀/ED₅₀. The dosage regimen will be determined by the attending physician and other clinical factors; preferably in accordance with any one of the above described methods. As is well known in the medical arts, dosages for any one patient depend upon many factors, which may include the patient's size, body surface area, age, the particular compound to be administered, sex, time and route of administration, general health, and other drugs being administered concurrently. Progress can be monitored by periodic assessment. A typical dose can be, for example, in the range of 1 μg to 1000 mg; however, doses below or above this exemplary range are envisioned, especially considering the aforementioned factors. Generally, the regimen as a regular administration of the pharmaceutical composition should be in the range of 1 μg to 100 mg units per day. If the regimen is a continuous infusion, it should also be in the range of 1 μg to 1 mg units per kilogram of body weight per minute, respectively. Preferably, the pharmaceutical composition is administered once to the subject, i.e., preferably, is used as a one-time treatment. Depending on the subject and the mode of administration, the quantity of substance administration may vary over a wide range to provide from about 0.01 mg per kg body mass to about 10 mg per kg body mass. The pharmaceutical compositions and formulations referred to herein are administered at least once in order to treat or ameliorate or prevent a disease or condition recited in this specification. However, the said pharmaceutical compositions may be administered more than one time, for example from two to 50 times, more preferably from five to 50 times. Preferably, administration is adjusted to maintain an effective concentration in the body of a subject for the time period intended, e.g. until surgical removal of one or more tumor(s) was performed. Also, as indicated above, the pharmaceutical preparation may be administered topically at the site of an excised tumor as a depot; in such case, the depot preferably is adjusted to maintain an effective dose until at least after additional cancer treatment, e.g. chemotherapy, was administered or, preferably, until such additional cancer treatment has been completed. Progress can be monitored by periodic assessment.

The term “preventing” an adverse health-related event, e.g. metastasis and/or tissue invasion in cancer, as used herein, refers to retaining health with respect to the adverse health-related event for a certain period of time in a subject. It will be understood that the said period of time is dependent on the amount of the active compound which has been administered and individual factors of the subject discussed elsewhere in this specification. It is to be also understood that prevention may not be effective in all subjects treated with the compound according to the present invention. However, the term preferably requires that a statistically significant portion of subjects of a cohort or population are effectively prevented from suffering from a disease or disorder referred to herein or its accompanying symptoms. Preferably, a cohort or population of subjects is envisaged in this context which normally, i.e. without preventive measures according to the present invention, would develop the adverse health-related event. Whether a portion is statistically significant can be determined without further ado by the person skilled in the art using various well known statistic evaluation tools, e.g., determination of confidence intervals, p-value determination, Student's t-test, Mann-Whitney test etc. Preferred confidence intervals are at least 90%, at least 95%, at least 97%, at least 98% or at least 99%. The p-values are, preferably, 0.1, 0.05, 0.01, 0.005, or 0.0001. Preferably, preventing shall be effective for at least 60%, at least 70%, at least 80%, or at least 90% of the subjects of a given cohort or population. Preferably, the above applies to “preventing cancer” mutatis mutandis.

The term “cancer” is, in principle, understood by the skilled person and relates to a disease of an animal, including man, characterized by uncontrolled growth by a group of body cells (“cancer cells”). This uncontrolled growth may be accompanied by intrusion into and destruction of surrounding tissue and possibly spread of cancer cells to other locations in the body. Preferably, also included by the term cancer is tumor recurrence, i.e. relapse. Thus, preferably, the cancer is a solid cancer, i.e. a cancer forming at least one detectable tumor, a metastasis, or a relapse thereof. Preferably, the cancer is selected from the list consisting of aids-related lymphoma, anal cancer, appendix cancer, astrocytoma, atypical teratoid, basal cell carcinoma, bile duct cancer, bladder cancer, brain stem glioma, breast cancer, burkitt lymphoma, carcinoid tumor, cerebellar astrocytoma, cervical cancer, chordoma, colon cancer, colorectal cancer, craniopharyngioma, endometrial cancer, ependymoblastoma, ependymoma, esophageal cancer, extracranial germ cell tumor, extragonadal germ cell tumor, extrahepatic bile duct cancer, fibrosarcoma, gallbladder cancer, gastric cancer, gastrointestinal stromal tumor, gestational trophoblastic tumor, head and neck cancer, hepatocellular cancer, hodgkin lymphoma, hypopharyngeal cancer, hypothalamic and visual pathway glioma, intraocular melanoma, kaposi sarcoma, laryngeal cancer, medulloblastoma, medulloepithelioma, melanoma, merkel cell carcinoma, mesothelioma, mouth cancer, multiple endocrine neoplasia syndrome, multiple myeloma, mycosis fungoides, nasal cavity and paranasal sinus cancer, nasopharyngeal cancer, neuroblastoma, non-hodgkin lymphoma, non-small cell lung cancer, oral cancer, oropharyngeal cancer, osteosarcoma, ovarian cancer, ovarian epithelial cancer, ovarian germ cell tumor, ovarian low malignant potential tumor, pancreatic cancer, papillomatosis, paranasal sinus and nasal cavity cancer, parathyroid cancer, penile cancer, pharyngeal cancer, pheochromocytoma, pituitary tumor, pleuropulmonary blastoma, primary central nervous system lymphoma, prostate cancer, rectal cancer, renal cell cancer, retinoblastoma, rhabdomyosarcoma, salivary gland cancer, sezary syndrome, small cell lung cancer, small intestine cancer, soft tissue sarcoma, squamous cell carcinoma, squamous neck cancer, testicular cancer, throat cancer, thymic carcinoma, thymoma, thyroid cancer, urethral cancer, uterine sarcoma, vaginal cancer, vulvar cancer, waldenstrom macroglobulinemia, and wilms tumor. More preferably, the cancer is brain cancer, colorectal cancer, breast cancer, pancreatic cancer, lung cancer, bladder cancer, prostate cancer, or ovarian cancer. Preferably, the cancer is a brain cancer, more preferably is a glioma, a meningioma, or an adenoma. More preferably, the cancer is a glioma, most preferably an astrocytoma such as glioblastoma multiforme, an ependymoma, or an oligodendroglioma. Preferably, the cancer cell is a mesenchymal cancer cell, preferably with low expression of E-cadherin and/or high expression of vimentin.

The term “migration of cancer cells” as used herein, relates to any type of at least partially active locomotion by a cancer cell, i.e. includes any type of change of location by a cancer cell which is not passive (e.g. via the blood stream). Preferably, tissue invasion by cancer cells and/or metastasis are caused by cancer cell migration. Preferably, migration of cancer cells comprises at least extracellular matrix invasion or active assembly of cytoskeleton components, in particular at the leading edge of the cancer cell, preferably actin and/or microtubule polymerization at the leading edge. Preferably, cancer cell migration is migration of a mesenchymal cancer cell, preferably with low expression of E-cadherin and/or high expression of vimentin. Methods of determining cancer cell migration are known to the skilled person; preferably, cancer cell migration is determined by a transwell assay known to the skilled person; or by determining migration of cancer cells into a film of extracellular matrix material, preferably extracellular matrix material secreted by Engelbreth-Holm-Swarm (EHS) mouse sarcoma cells, which is commercially available under the designations Matrigel™ or Cultrex BME™. Preferably, cancer cell migration is determined as specified herein in the Examples.

In accordance with the above, the term “preventing cancer cell migration” relates to a statistically significant reduction of cancer cell migration, preferably by at least 50%, more preferably at least 75%, still more preferably at least 90%, even more preferably at least 95%. Preferably, preventing cancer cell migration comprises reducing the frequency of metastasis formation, reducing the frequency of relapse, and/or reducing the extent of tissue invasion by a tumor, preferably by at least 30%, more preferably at least 50%, still more preferably at least 75%, even more preferably at least 90%. Also preferably, preventing migration of cancer cells is reducing circulating cancer cell count (CTC) in a bodily fluid sample by at least a factor of 2, preferably at least a factor of five, more preferably at least a factor of ten, most preferably at least a factor of 25, preferably, the bodily fluid is blood or liquor in such case. Most preferably, preventing cancer cell migration comprises abolishment of cancer cell migration, metastasis formation and/or tissue invasion. Preferably, preventing migration of cancer cells does not comprise killing of cancer cells, preferably wherein significant killing of cancer cells is killing of more than 30% of cancer cells within 3 days, more preferably more than 20% within 3 days, most preferably more than 10% within 3 days; more preferably, preventing migration of cancer cells does not comprise killing of cancer cells. The effect of preventing cancer cell migration can be determined by the above-referenced methods for determining cancer cell migration. In accordance with the above, preventing cancer cell migration may, however, also be determined in vivo in a group of subjects suffering from cancer by determining the frequency of metastasis formation and/or of relapse, and/or the extent of tissue invasion. Preferably, preventing cancer cell migration comprises induction of mesenchymal-epithelial transition (MET) in cancer cells; accordingly, preventing cancer cell migration may also be determined by surrogate markers, such as molecular markers of mesenchymal cells and/or molecular markers of epithelial cells, in particular E-cadherin and/or vimentin.

The term “treating cancer”, preferably refers to an amelioration of a cancer referred to herein or the symptoms accompanied therewith, preferably to a significant extent. Said treating as used herein preferably also includes an entire restoration of the health with respect to the diseases or disorders referred to herein. It is to be understood that treating as used in accordance with the present invention may not be effective in all subjects to be treated. However, the term shall require that, preferably, a statistically significant portion of subjects suffering from a disease or disorder referred to herein can be successfully treated. Whether a portion is statistically significant can be determined without further ado by the person skilled in the art using various well known statistic evaluation tools, in particular as specified herein above.

The term “subject”, as used herein, relates to a multicellular animal, preferably to a vertebrate, more preferably to a mammal. More preferably, the subject is a human, a cattle, a pig, a sheep, a goat, a horse, a cat, a dog, a guinea pig, a mouse, or a rat. Preferably, the subject is a laboratory animal, preferably a guinea pig, a mouse, or a rat. Also preferably, the subject is a livestock, preferably a cattle, a pig, a sheep, a goat, or a horse. Also preferably, the subject is a companion animal, preferably a cat, a dog, or a guinea pig. Most preferably, the subject is a human. Preferably, the subject is known or suspected to suffer from cancer. More preferably, the subject is suspected to suffer from cancer, preferably based on a preliminary diagnosis, preferably a biochemical marker screening. Also more preferably, the subject is known to suffer from cancer, preferably from a cancer type known in the art to have a high tissue invasion potential and/or metastatic potential. Still more preferably, the subject is known to suffer from a cancer type known in the art to have a high tissue invasion potential and also known to have a high risk of misadjusting safety margins during excision; examples of the latter cancer type are gliomas, in particular glioblastomas such as glioblastoma multiforme. As the skilled person will understand in the light of the present specification, in a subject suspected to suffer from cancer, the composition described herein may be used to prevent metastasis formation and/or (further) tissue invasion by a potential tumor; thus, the composition preferably will be administered in a manner to prevent metastasis formation and tissue formation, i.e. preferably, systemically. As the skilled person will also understand in the light of the present specification, in a subject known to suffer from a cancer type known in the art to have a high tissue invasion potential and also known to have a high risk of misadjusting safety margins during excision, such as glioma, in particular glioblastoma such as glioblastoma multiforme, the composition as specified herein may preferably be administered at a site of tumor excision, to prevent (further) tissue invasion and relapse, and may preferably administered topically, preferably as a depot. Also preferably, in the subject, the cancer has formed at most ten, preferably at most five, more preferably at most two, most preferably one, detectable tumor(s). Also preferably, in the subject, the largest of said tumor(s), preferably the primary tumor, has a size of at most 25 mm, preferably of at most 10 mm, still more preferably at most 5 mm.

Preferably, preventing cancer cell migration is accompanied by administration of cancer therapy. The term “cancer therapy”, as used herein, relates to measures administered to a subject to remove cancer cells from the subject, to kill cancer cells in the subject, to inhibit growth of cancer cells in the subject, and/or to cause the body of the subject to inhibit growth of or to kill cancer cells. Thus, cancer therapy, preferably is surgery, radiotherapy, chemotherapy, anti-hormone therapy, targeted therapy, and/or immunotherapy. Preferably, cancer therapy comprises surgery; more preferably, cancer therapy comprises surgery and radiotherapy; still more preferably, cancer therapy comprises surgery, radiotherapy, and chemotherapy. Cancer therapy may be administered before, simultaneously to, and/or after preventing migration of cancer cells.

The terms “radiation therapy” and “radiotherapy” are known to the skilled artisan. The terms relate to the use of ionizing radiation to treat or control cancer. The skilled person also knows the term “surgery”, relating to invasive measures for treating cancer, in particular excision of tumor tissue.

As used herein, the term “chemotherapy” relates to treatment of a subject with an antineoplastic drug. Preferably, chemotherapy is a treatment including alkylating agents (e.g. cyclophosphamide), platinum (e.g. carboplatin), anthracyclines (e.g. doxorubicin, epirubicin, idarubicin, or daunorubicin) and topoisomerase II inhibitors (e.g. etoposide, irinotecan, topotecan, camptothecin, or VP16), anaplastic lymphoma kinase (ALK)-inhibitors (e.g. Crizotinib or AP26130), aurora kinase inhibitors (e.g. N-[4-[4-(4-Methylpiperazin-1-yl)-6-[(5-methyl-1H-pyrazol-3-yl)amino]pyrimidin-2-yl]sulfanylphenyl]cyclopropanecarboxamide (VX-680)), antiangiogenic agents (e.g. Bevacizumab), or Iodine131-1-(3-iodobenzyl)guanidine (therapeutic metaiodobenzylguanidine), histone deacetylase (HDAC) inhibitors, alone or any suitable combination thereof. It is to be understood that chemotherapy, preferably, relates to a complete cycle of treatment, i.e. a series of several (e.g. four, six, or eight) doses of antineoplastic drug or drugs applied to a subject separated by several days or weeks without such application.

The term “anti-hormone therapy” relates to cancer therapy by blocking hormone receptors, e.g. estrogen receptor or progesterone receptor, expressed on cancer cells, or by blocking the biosynthesis of a hormone the cancer is dependent on. Blocking of hormone receptors can preferably be achieved by administering compounds, e.g. tamoxifen, binding specifically and thereby blocking the activity of said hormone receptors. Blocking of hormone biosynthesis is preferably achieved by administration of inhibitors, e.g. in the case of estrogen, aromatase inhibitors like, e.g. anastrozole or letrozole may be used. It is known to the skilled artisan that anti-hormone therapy is only advisable in cases where tumor cells are expressing hormone receptors.

The term “targeted therapy”, as used herein, relates to application to a patient of a chemical substance known to block growth of cancer cells by interfering with specific molecules known to be necessary for tumorigenesis or cancer or cancer cell growth. Examples known to the skilled artisan are small molecules like, e.g. PARP-inhibitors (e.g. Iniparib), or monoclonal antibodies like, e.g., Trastuzumab.

The term “immunotherapy” as used herein relates to the treatment of cancer by modulation of the immune response of a subject. Said modulation may be inducing, enhancing, or suppressing said immune response. The term “cell based immunotherapy” relates to a cancer therapy comprising application of immune cells, e.g. T-cells, preferably tumor-specific NK cells, to a subject.

Advantageously, it was found in the work underlying the present invention that the compounds and compositions specified herein have the biological effect of preventing cancer cells from migrating, making them suitable in prevention of metastasis formation, tissue invasion, and relapse.

The definitions made above apply mutatis mutandis to the following. Additional definitions and explanations made further below also apply for all embodiments described in this specification mutatis mutandis.

The present invention further relates to a combined preparation comprising a composition of the present invention and a cancer therapeutic agent.

The term “combined preparation”, as referred to in this application, relates to a preparation comprising the pharmaceutically active compounds of the present invention in one preparation. Preferably, the combined preparation is comprised in a container, i.e. preferably, said container comprises all pharmaceutically active compounds of the present invention. Preferably, said container comprises the pharmaceutically active compounds of the present invention as separate formulations, i.e. preferably, one formulation of the active compound as specified herein and one formulation of a cancer therapeutic agent. As will be understood by the skilled person, the term “formulation” relates to a, preferably pharmaceutically acceptable, mixture of compounds, comprising or consisting of at least one pharmaceutically active compound. Preferably, the combined preparation comprises active compound and the further active compound in a single solid pharmaceutical form, e.g. a tablet or, preferably, a solution; more preferably, the active compounds of the present invention are comprised in two separate, preferably liquid, formulations; said separate liquid formulations, preferably are for injection, preferably at different parts of the body of a subject.

Preferably, the combined preparation is for separate or for combined administration. “Separate administration”, as used herein, relates to an administration wherein at least two active compounds are administered via different routes and/or at different parts of the body of a subject. E.g. one compound may be administered by enteral administration (e.g. orally), whereas a second compound is administered by parenteral administration (e.g. intravenously). Preferably, the combined preparation for separate administration comprises at least two physically separated preparations for separate administration, wherein each preparation contains at least one active or further active compound; said alternative is preferred e.g. in cases where the pharmaceutically active compounds of the combined preparation have to be administered by different routes, e.g. parenterally and orally, due to their chemical or physiological properties. Conversely, “combined administration” relates to an administration wherein the active compounds are administered via the same route, e.g. orally or, preferably, intravenously.

Also preferably, the combined preparation is for simultaneous or for sequential administration. “Simultaneous administration”, as used herein, relates to an administration wherein the active compounds are administered at the same time, i.e., preferably, administration of the pharmaceutically active compounds starts within a time interval of less than 15 minutes, more preferably, within a time interval of less than 5 minutes. Most preferably, administration of the pharmaceutically active compounds starts at the same time, e.g. by swallowing a tablet comprising the pharmaceutically active compounds, or by swallowing a tablet comprising one of the pharmaceutically active compounds and simultaneous injection of the second compound, or by administering an intravenous injection of a solution comprising an active compound and injecting a further active compound in a different part of the body, or by administering an intravenous injection of a solution comprising the active compound and the further active compound. Conversely, “sequential administration”, as used herein, relates to an administration causing plasma concentrations of the active compounds in a subject enabling the synergistic effect of the present invention, but which, preferably, is not a simultaneous administration as specified herein above. Preferably, sequential administration is an administration wherein administration of the active compounds, preferably all active compounds, starts within a time interval of 1 or 2 days, more preferably within a time interval of 12 hours, still more preferably within a time interval of 4 hours, even more preferably within a time interval of one hour, most preferably within a time interval of 5 minutes.

The term “cancer therapeutic agent” relates to an agent, preferably a pharmaceutic compound used in cancer therapy as specified herein above. Thus, preferably, the cancer therapeutic agent is a chemotherapeutic agent, an anti-hormone therapeutic agent, targeted therapeutic agent, and an immunotherapeutic agent, all preferably as specified herein above. Preferably, the cancer therapeutic agent is an agent inducing cell death in cells of said cancer, more preferably a chemotherapeutic agent.

The present invention also relates to a combined preparation according to the present invention for use in medicine; and to a combined preparation according to the present invention for use in treating cancer in a subject known or suspected to suffer from cancer.

In the cancer treatment with the combined preparation, the subject preferably is known to suffer from cancer, more preferably was diagnosed to have at least one detectable tumor. Also preferably, the cancer treatment comprises preventing metastasis and tumor removal, e.g. by surgery. More preferably, the cancer treatment comprises preventing metastasis, tumor removal and/or killing of cancer cells, wherein killing of cancer cells is preferably achieved by one of the cancer therapies specified herein above.

The present invention also relates to a method for preventing migration or invasion of cancer cells, comprising contacting cancer cells with a composition of the present invention, and thereby preventing migration or invasion of cancer cells.

The method of the present invention, may be an in vivo or an in vitro method. Moreover, it may comprise steps in addition to those explicitly mentioned above. For example, further steps may relate, e.g., to diagnosing cancer in a subject in case of an in vivo method, or to providing a sample of cancer cells in case of an in vitro method. Moreover, one or more of said steps may be performed by automated equipment.

As the skilled person will understand, in case the method of the present invention is an in vivo method, i.e. a method of treatment or part thereof, the specification elsewhere herein relating to prevention and treatment is applicable mutatis mutandis; thus, preferably, the aforesaid method is part of a method for treating cancer, comprising the steps of the method for preventing migration of cancer cells and administering at least one anticancer therapy, preferably as specified herein above. In case the method is an in vitro method, it is preferably performed on isolated cells, which are, preferably, not returned into the body of the subject they originate from, more preferably are not returned into the body of a subject. Thus, the in vitro method preferably is used for cell culture in which migration and/or invasion of cancer cells is undesirable; thus, the method may e.g. be used as a negative control, i.e. non-migrating and/or non-invading control, in migration and/or invasion experiments. In case the method is an in vitro method, tissue invasion preferably relates to invasion in an in vitro tissue and/or invasion model, preferably as specified herein above and/or in the Examples.

In accordance with the above, the present invention also relates to a use of a compound as specified elsewhere herein, in particular a metal complex as specified, for in vitro prevention of migration and/or tissue invasion of cancer cells.

The present invention also relates to a kit comprising a compound as specified elsewhere herein, in particular a metal complex as specified, and a pharmaceutically acceptable carrier for use in preventing migration of cancer cells in a subject known or suspected to suffer from cancer.

The term “kit”, as used herein, refers to a collection of the aforementioned components. Preferably, said components are combined with additional components, preferably within an outer container. Examples for such components of the kit as well as methods for their use have been given elsewhere in this specification. The kit, preferably, contains the aforementioned components in a ready-to-use formulation. The kit preferably comprises further components, preferably a cancer therapeutic agent as specified herein above, a diluent, and/or a means of administration, in particular a syringe and/or a needle, and/or an IV infusion equipment. Preferably, the kit may additionally comprise instructions, e.g., a user's manual for carrying out a method of the present invention. Details are to be found elsewhere in this specification. Additionally, such user's manual may provide instructions about correctly using the components of the kit. A user's manual may be provided in paper or electronic form, e.g., stored on CD or CD ROM. The present invention also relates to the use of said kit in any of the methods according to the present invention.

The present invention also relates to a use of a compound as specified elsewhere herein, in particular a metal complex as specified, in the manufacture of a pharmaceutic preparation for preventing migration of cancer cells.

The present invention also relates to a method, preferably an in vitro method, for determining whether cancer cells are susceptible to immobilizing by a composition according to the present invention, comprising contacting said cancer cells to said composition and determining cancer cells to be susceptible to immobilizing by said compound in case the cancer cells are found to be immobilized.

The method for determining whether cancer cells are susceptible to immobilizing preferably is an in vitro method and may comprise steps in addition to those mentioned above. E.g. additional steps may relate to providing a sample of cancer cells, preferably from a subject, or to determining after said contacting whether the cancer cells are immobilized. As the skilled person will understand, the method for determining whether cancer cells are susceptible to immobilizing preferably is a companion diagnostic method, i.e. a method preferably performed on a sample of a cancer, e.g. a biopsy or an excised tumor or fraction thereof, in order to determine sensitivity of cancer cells to the compounds of the present invention. As will also be understood, the same method may also be used to determine an effective dose of the compounds with regards to the specific cancer cells.

The term “contacting” as used in the context of the methods of the present invention is understood by the skilled person. Preferably, the term relates to bringing a compound or composition of the present invention in physical contact with a subject or cell and thereby allowing said subject or cell and the compound or composition to interact. As the skilled person will understand, in as far as the term “contacting” relates to contacting a cell of a subject with a compound of the present invention, contacting may be administering compound to said subject as specified herein above.

The term “sample”, as used herein, refers to any sample suspected or known to comprise cancer cells; thus, the sample preferably is a biological sample. Preferably, the sample is a sample of a body fluid, a sample of separated cells, a sample from a tissue or an organ, or a sample of wash/rinse fluid obtained from an outer or inner body surface of a subject. Preferably, the sample is a body fluid like blood, plasma, serum, urine, saliva, or lacrimal fluid. More preferably, the sample is a tissue, more preferably a tumor sample, known or suspected to comprise cancer cells. Samples can be obtained by well-known techniques which include, preferably, scrapes, swabs or biopsies. Such samples can be obtained by use of brushes, (cotton) swabs, spatula, rinse/wash fluids, punch biopsy devices, puncture of cavities with needles or by surgical instrumentation. Preferably, the sample is a biopsy, a tumor, or part thereof, of a cancer as specified elsewhere herein. Separated cells and/or cell-free liquids may be obtained from cell culture supernatants, body fluids, or the tissues or organs by separating techniques such as filtration, centrifugation, or cell sorting. It is to be understood that the sample may be further processed in order to carry out a method of the present invention.

All references cited in this specification are herewith incorporated by reference with respect to their entire disclosure content and the disclosure content specifically mentioned in this specification.

In view of the above, the following embodiments are particularly envisaged:

Embodiment 1. A composition for use in preventing migration of cancer cells in a subject known or suspected to suffer from cancer, the composition comprising at least one metal complex having the structure (I)

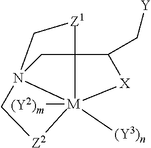

wherein

M is a metal, preferably selected from the group consisting of copper, iron, manganese and zinc, X is X^{a} or X^{b}, wherein X^{a} is selected from the group consisting of O, S and —N(R¹)—, wherein R¹ is H or alkyl, and wherein X^{b} is a group forming a coordinate covalent bond to a second metal M′, preferably a group O, S or —N(R¹)—, wherein M′ is preferably selected from the group consisting of copper, iron, manganese and zinc, and wherein M′ and M may be the same or different and are preferably the same,

Z¹ and Z², are independently of each other a, substituted or unsubstituted, -Aryl-O—, -Aryl-N— or heteroaryl group, Y is Y^{a} or Y^{b}, wherein Y^{a} is selected from the group consisting of H, alkyl, —OH, —SH, halogen, and —NR³R⁴, wherein R³ and R⁴, are independently of each other selected from H and alkyl, preferably R³ and R⁴ are both H, and wherein Y^{b} is a group forming a coordinate covalent bond to M or M′, preferably Y^{b} is a group O, S or —N(R^{Yb1}R^{Yb2}), wherein R^{Y}bi and R^{Yb1} are, independently of each other, H or alkyl, preferably H, and wherein n and m are integers, which are independently of each other, 0 or 1, Y² is water or a halogen, and wherein Y³ is water or a halogen.

Embodiment 2. The composition for use of embodiment 1, wherein the complex is a mononuclear or binuclear complex or a mixture thereof.

Embodiment 3. The composition for use of embodiment 1 or 2, wherein X is X^{a}.

Embodiment 4. The composition for use of any one of embodiments 1 to 3, wherein X is X^{b}.

Embodiment 5. The composition for use of any one of embodiments 1 to 4, wherein Y is Y^{a}, wherein Y^{a} is preferably selected from the group consisting of —Cl and —NH₂.

Embodiment 6. The composition for use of any one of embodiments 1 to 5, wherein Y is Y^{b}, wherein Y^{b} is preferably —NH₂.

Embodiment 7. The composition for use of any one of embodiments 1 to 6, wherein Y is Y^{a} and wherein the complex has one of the following structures:

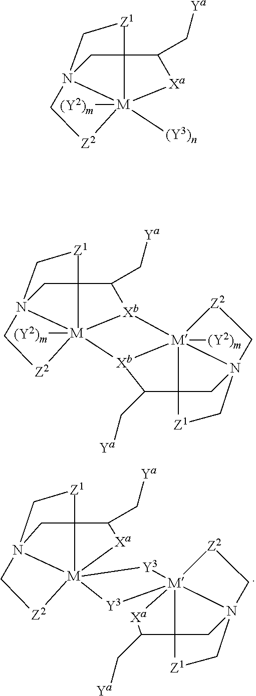

Embodiment 8. The composition for use of any one of embodiments 1 to 7, wherein Y is Y^{b} and wherein the complex has the structure

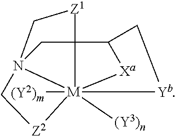

Embodiment 9. The composition for use of any one of embodiments 1 to 8, wherein M and M′ are, independently of each other selected from the group consisting of Fe(III), Cu(II), Mn(II) and Zn(II), with Fe(III) and Cu(II) being particularly preferred.

Embodiment 10. The composition for use of any one of embodiments 1 to 9, wherein Z1 is a substituted or unsubstituted, -Aryl-O—, preferably a -phenyl-O— group, thus a group having the structure

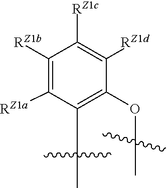

with R^{Z1a}, R^{Z1b}, R^{Z1c} and R^{Z1d}, being, independently of each other, selected from the group consisting of H and alkyl.

Embodiment 11. The composition for use of any one of embodiments 1 to 10, wherein the complex has a structure selected from the group consisting of

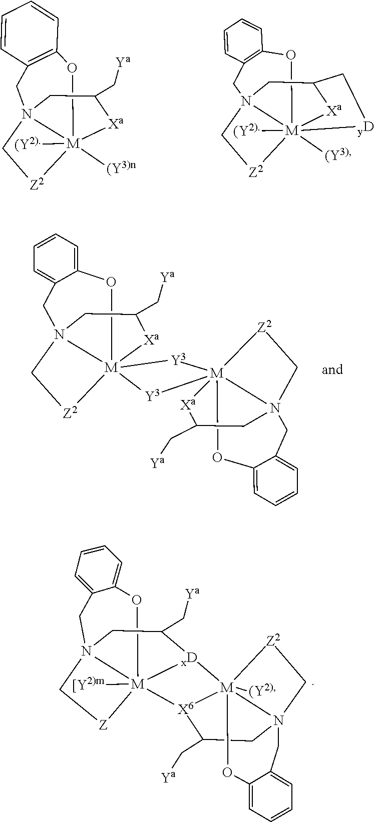

Embodiment 12. The composition for use of any one of embodiments 1 to 10, wherein Z2 is a substituted or unsubstituted pyridyl group, thus a group having the structure,

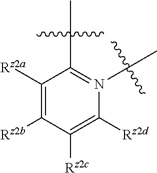

with R^{Z2a}, R^{Z2b}, R^{Z2c} and R^{Z2d} being, independently of each other, selected from the group consisting of H, optionally substituted, alkyl, and, optionally substituted, aryl.

Embodiment 13. The composition for use of any one of embodiments 1 to 12, wherein the complex has a structure selected from the group consisting of

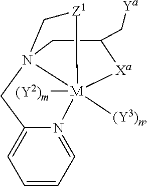

preferably

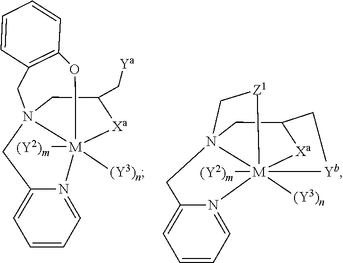

preferably

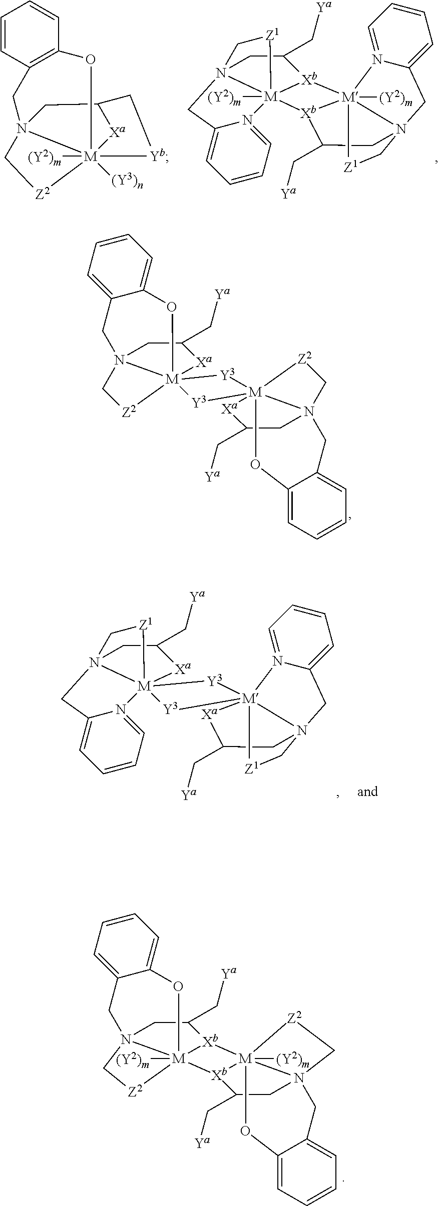

Embodiment 14. The composition for use of any one of embodiments 1 to 13, wherein the complex has a structure selected from the group consisting of

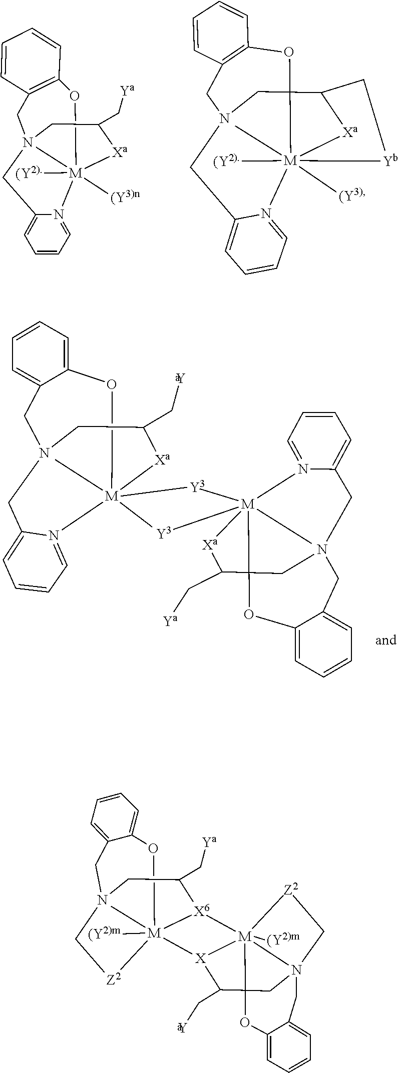

Embodiment 15. The composition for use of any one of embodiments 1 to 14, wherein X is wherein X is —O—.

Embodiment 16. The composition for use of any one of embodiments 1 to 15, wherein Y is —Cl or —NH₂, more preferably —Cl.

Embodiment 17. The composition for use of any one of embodiments 1 to 16, wherein the complex has as structure selected from the group consisting of.

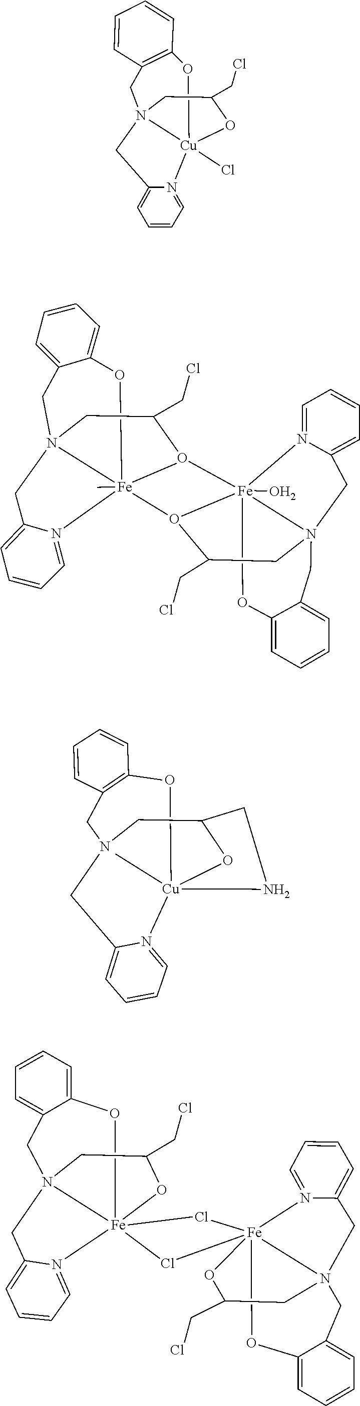

and mixtures of two or more thereof.

Embodiment 18. Composition for use in preventing migration of cancer cells in a subject known or suspected to suffer from cancer, the composition comprising a metal complex, wherein the metal complex is obtained or obtainable by reacting a metal salt comprising a metal ion M* with a ligand having the structure (I*)

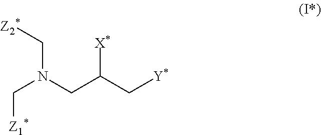

wherein X* is selected from the group consisting of —OH, SH, —N(R¹)(R²)—, wherein R¹ is H or alkyl, and wherein R² is H or alkyl; Z^{1*} and Z^{2*} are independently of each other, a substituted aryl or a, substituted or unsubstituted, heteroaryl group; wherein the substituted aryl group is preferably substituted with an hydroxyl or amine group, Y* is selected from the group consisting of H, alkyl, —OH, —SH, halogen and —NR³R⁴; and wherein M* is selected from the group consisting of copper, iron, magnesium, and manganese.

Embodiment 19. The composition for use of embodiment 18, wherein the ligand is selected from the group consisting of

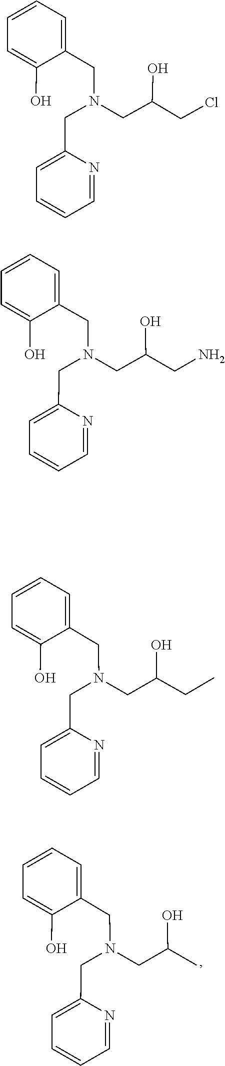 [SMILES: Oc1ccccc1CN(Cc1ccccn1)CC(O)CCl ; NCC(O)CN(Cc1ccccn1)Cc1ccccc1O ; CCC(O)CN(Cc1ccccn1)Cc1ccccc1O ; CC(O)CN(Cc1ccccn1)Cc1ccccc1O]

preferably wherein the ligand is

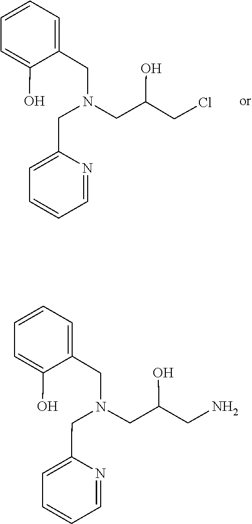 [SMILES: Oc1ccccc1CN(Cc1ccccn1)CC(O)CCl ; NCC(O)CN(Cc1ccccn1)Cc1ccccc1O]

more preferably, wherein the ligand is

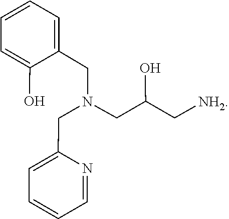 [SMILES: NCC(O)CN(Cc1ccccn1)Cc1ccccc1O]

Embodiment 20. The composition for use of any one of embodiments 1 to 19, wherein said preventing migration of cancer cells is preventing metastasis, tissue invasion, and/or relapse.

Embodiment 21. The composition for use of any one of embodiments 1 to 20, wherein said preventing cancer cell migration comprises reducing the frequency of metastasis formation, reducing the frequency of relapse, and/or reducing the extent of tissue invasion by a tumor by at least 30%, preferably at least 50%, more preferably at least 75%, most preferably at least 90%.

Embodiment 22. The composition for use of any one of embodiments 1 to 21, wherein said preventing migration of cancer cells does not comprise significant killing of cancer cells.

Embodiment 23. The composition for use of any one of embodiments 1 to 22, wherein said cancer is a solid cancer.

Embodiment 24. The composition for use of any one of embodiments 1 to 23, wherein said cancer has formed at least one detectable tumor in the subject.

Embodiment 25. The composition for use of any one of embodiments 1 to 24, wherein said cancer has formed at most ten, preferably at most five, more preferably at most two, most preferably one, detectable tumor(s) in the subject.

Embodiment 26 The composition for use of embodiment 25, wherein the largest of said tumor(s), preferably the primary tumor, has a size of at most 25 mm.

Embodiment 27. The composition for use of any one of embodiments 1 to 26, wherein said composition is administered before tumor removal and/or wherein said composition is administered at a site of tumor removal.

Embodiment 28. The composition for use of any one of embodiments 1 to 27, wherein said subject is suspected to suffer from cancer, preferably based on a biochemical marker screening.

Embodiment 29. The composition for use of any one of embodiments 1 to 28, wherein said cancer is brain cancer, colorectal cancer, breast cancer, pancreatic cancer, lung cancer, bladder cancer, prostate cancer, or ovarian cancer, preferably is brain cancer, more preferably glioma.

Embodiment 30. The composition for use of any one of embodiments 1 to 29, wherein said cancer is a glioma, preferably a glioblastoma.

Embodiment 32. The composition for use of any one of embodiments 1 to 30, wherein said composition is a pharmaceutically compatible composition.

Embodiment 33. A composition as specified in any one of embodiments 1 to 19 for use in treating cancer by a cancer therapy, wherein said treating cancer comprises preventing metastasis and/or tissue invasion.

Embodiment 34. A combined preparation comprising a composition as specified in any one of embodiments 1 to 19 and a cancer therapeutic agent.

Embodiment 35. The combined preparation of embodiment 34, wherein said cancer therapeutic agent is at least one of a chemotherapeutic agent, an anti-hormone therapeutic agent, a targeted therapeutic agent, and an immunotherapeutic agent.

Embodiment 36. The combined preparation of embodiment 34 or 35, wherein said cancer therapeutic agent is an agent inducing cell death in cells of said cancer.

Embodiment 37. The combined preparation of any one of embodiments 34 to 36, wherein said combined preparation is a combined preparation for simultaneous, separate or sequential use.

Embodiment 38. A combined preparation according to any one of embodiments 34 to 37 for use in medicine.

Embodiment 39. A combined preparation according to any one of embodiments 34 to 37 for use in treating cancer in a subject known or suspected to suffer from cancer.

Embodiment 40. The combined preparation for use of embodiment 39, wherein said cancer treatment comprises preventing metastasis and/or relapse.

Embodiment 41. The combined preparation for use of embodiment 39 or 40, wherein said cancer has formed at least one detectable tumor in the subject.

Embodiment 42. The combined preparation for use of any one of embodiments 39 to 40, wherein said cancer treatment comprises preventing metastasis and tumor removal.

Embodiment 43. The combined preparation for use of any one of embodiments 39 to 40, wherein said cancer treatment comprises preventing metastasis and tumor removal and/or killing of cancer cells.

Embodiment 44. A method for preventing migration of cancer cells, comprising contacting cancer cells with a compound as specified in any one of embodiments 1 to 19, and thereby preventing migration of cancer cells.

Embodiment 45. A method for treating cancer, comprising the steps of the method for preventing migration of cancer cells according to embodiment 44 and administering at least one anticancer therapy.

Embodiment 46. The method of embodiment 45, wherein said anticancer therapy is selected from the list consisting of (i) radiotherapy, (ii) chemotherapy, (iii) anti-hormone therapy, (iv) targeted therapy, (v) immunotherapy, and (vi) any combination of (i) to (v).

Embodiment 47. A kit comprising a compound as specified in any one of embodiments 1 to 19 and a pharmaceutically acceptable carrier for use in preventing migration of cancer cells in a subject known or suspected to suffer from cancer.

Embodiment 48. The kit of embodiment 47, further comprising at least one of a chemotherapeutic agent, an anti-hormone therapeutic agent, a targeted therapeutic agent, and an immunotherapeutic agent.

Embodiment 49. Use of a compound as specified in any one of embodiments 1 to 19 in the manufacture of a pharmaceutic preparation for preventing migration of cancer cells.

Embodiment 50. Use of a compound as specified in any one of embodiments 1 to 19 for, preferably in vitro, prevention of migration and/or tissue invasion of cancer cells.

Embodiment 51. A method, preferably an in vitro method, for determining whether cancer cells are susceptible to immobilizing by a composition as specified in any one of embodiments 1 to 19, comprising contacting said cancer cells to said composition and determining cancer cells to be susceptible to immobilizing by said compound in case the cancer cells are found to be immobilized.

Embodiment 52. The subject matter of any of the preceding embodiments, wherein said composition induces a mesenchymal-epithelial transition in said cancer cells.

Embodiment 53: Composition according to embodiment 18, the metal complex being obtained or obtainable by a process comprising
(i) mixing a metal salt comprising a metal ion M* with a ligand having the structure

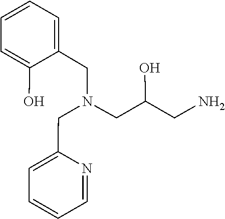 [SMILES: NCC(O)CN(Cc1ccccn1)Cc1ccccc1O]

preferably a copper salt, more preferably CuCl₂, more preferably in an organic solvent
(ii) precipitating the complex and isolating the complex. wherein (i) is preferably carried at elevated temperature, preferably under reflux.

Embodiment 54: Composition for use in preventing migration of cancer cells in a subject known or suspected to suffer from cancer, the composition comprising at least one metal complex, the complex having a structure selected from the group consisting of:

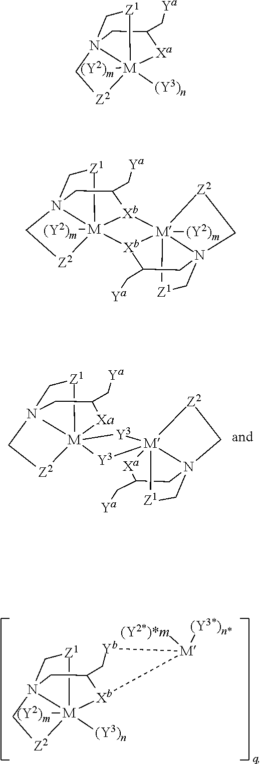

preferably

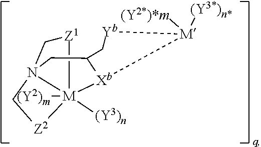

wherein
M and M′ are metals, preferably selected from the group consisting of copper, iron, manganese and zinc, wherein M′ and M may be the same or different and are preferably the same;
X^{b} is a group O, S or —N(R¹)—, wherein R¹ is H or alkyl;
Z¹ and Z², are independently of each other a, substituted or unsubstituted, -Aryl-O—, -Aryl-N— or heteroaryl group;
Y^{b} is a group O, S or —N(R^{Yb1}R^{Yb2}), wherein R^{Y}bi and R^{Y}bi are, independently of each other, H or alkyl, preferably H;
Y² is water or a halogen;
Y^{2*} is a solvent molecule or a halogen, preferably water, methanol or a halogen, more preferably, water, methanol or —Cl;
Y³ is water or a halogen;
Y^{3*} is a solvent molecule or a halogen, preferably water, methanol or a halogen, more preferably, water, methanol or —Cl;
n and m are integers, which are independently of each other, 0 or 1;
n* and m* are integers, which are independently of each other, 0 or 1; and
q is an integer of from 2 to 5, preferably q is 2 or 3, more preferably 2.

Embodiment 55: Composition according to embodiment 54, wherein the complex has the structure

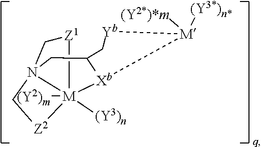

Embodiment 56: Composition according to embodiment 55, wherein q is 2 or 3, more preferably 2.

Embodiment 57: Composition according to embodiment 55, wherein q is 4.

Embodiment 58: Composition according to any one of embodiments 55 to 57, wherein m and n are 0.

Embodiment 59: Composition according to any one of embodiments 55 to 58, the complex

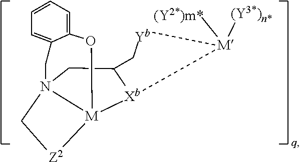

having the structure

more preferably, the structure

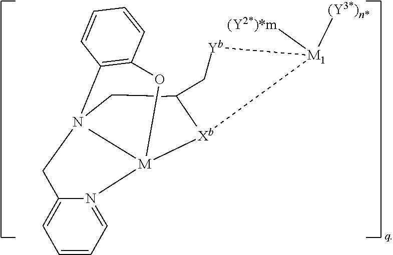

Embodiment 60: The composition according to embodiment 59, wherein q is 2, the complex having the structure

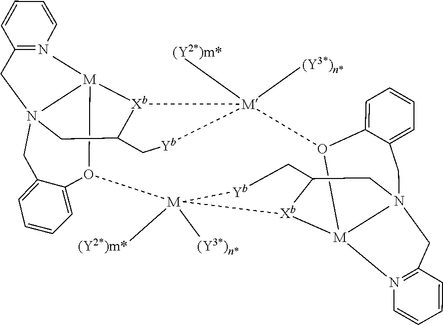

Embodiment 61: The composition according to any one of embodiments 54 to 60, wherein M and M′ are Cu.

Embodiment 62: The composition according to any one of embodiments 54 to 61, wherein Y^{b} is NH₂.

Embodiment 63: The composition according to any one of embodiments 54 to 62, wherein X^{b} is OH.

Embodiment 64: The composition according to any one of embodiments 54 to 63, wherein n* and m* are both 1.

Embodiment 65: Complex having the structure:

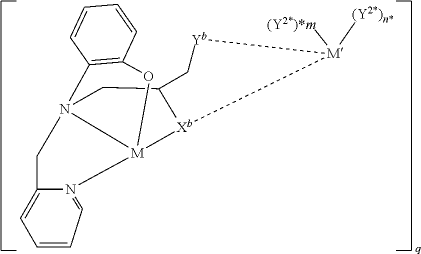

wherein with M and M′ are copper, Y^{b} is NH₂, X^{b} is a group forming a coordinate covalent bond to a second metal M′, preferably a group O, S or —N(R¹)—, more preferably O, and wherein m* and n* are both 1, wherein Y^{2*} is a solvent molecule or a halogen, preferably water, methanol or a halogen, more preferably, water, methanol or —Cl, and wherein Y^{3*} is a solvent molecule or a halogen, preferably water, methanol or a halogen, more preferably, water, methanol or —Cl, in particular wherein Y^{2*} and Y3* are both —Cl, and wherein q is an integer in the range of from 2 to 5.

Embodiment 66: The complex according to embodiment 66, wherein q is 2, the complex having the structure

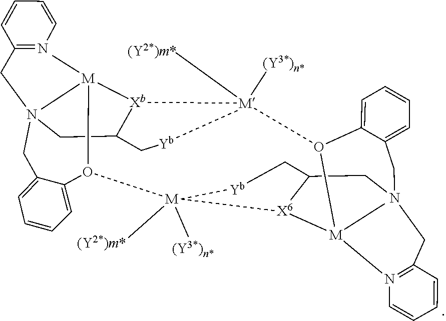

Embodiment 67: The complex according to embodiment 65 or 66, wherein X^{b} is OH.

Embodiment 68: The complex according to any one of embodiments 65 to 68, wherein Y³* and Y2* both —Cl.

Embodiment 69: Copper complex, the complex comprising the ligand

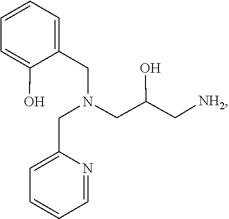 [SMILES: NCC(O)CN(Cc1ccccn1)Cc1ccccc1O]

wherein the complex is preferably obtained or obtainable upon reaction of CuCl₂, with the ligand and wherein the complex preferably displays a ratio of Cu:ligand:Cl of 2:1:1, and wherein the complex more preferably has the structure:

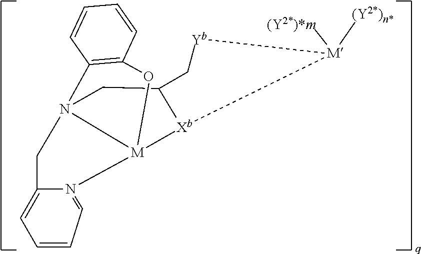

wherein with M and M′ are copper, Y^{b} is NH₂, X^{b} O, and wherein m* and n* are both 1, wherein Y^{2*} and Y^{3*} are, independently of each other selected from the group consisting of a solvent molecule, water and —Cl, more preferably both, Y^{2*} and Y^{3*} are preferably Cl, and wherein q is an integer in the range of from 2 to 5, preferably 2 or 3, more preferably 2

Embodiment 70: Complex according to any one of embodiments 65 to 69 for use as a medicament, preferably for use in preventing or treating cancer, more preferably for use in preventing migration of cancer cells in a subject known or suspected to suffer from cancer.

Embodiment 71: Compound having the structure:

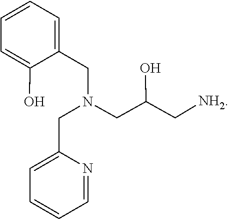 [SMILES: NCC(O)CN(Cc1ccccn1)Cc1ccccc1O]

Embodiment 72: Composition comprising a complex according to any one of embodiments 56 to 64 or complex according to any one of embodiments 65 to 70, wherein the complex is water soluble.

Embodiment 73: Composition comprising a complex according to any one of embodiments 56 to 64 or complex according to any one of embodiments 65 to 70, wherein the complex is water soluble up to a concentration of 2 mM, preferably as 1 mM.

### FIGURE LEGENDS

FIG. 1: Synthesis scheme of the ligand.

FIG. 2: Synthesis scheme of the complexes (FeL and CuL).

FIG. 3: Synthesis route for ligand (8).

FIG. 4: FeL and CuL complexes inhibit migration and induce mesenchymal-epithelial transition (MET) in H4 glioma cells. (A) Migration of H4 cells after 24 h of incubation with 25 μM of FeL and CuL assessed with the transwell migration assay. (B) Cell cycle analysis and (C) expression of EMT marker genes in H4 cells under those same conditions. The results were calculated from three independent experiments and are given as the mean±S.E.M. Statistical significance was calculated using one-way ANOVA, followed by Dunnet's test ((*P<0.05, **P<0.01).

FIG. 5: FeL and CuL complexes inhibit H4 spheroids invasion. Viability (A) and growth (B) of H4 spheroids after 24 h and 72 h of incubation with 25 μM of FeL and CuL. Invasion of H4 spheroids after 24 h and 72 h of incubation with 25 μM of FeL and CuL without (C) or with irradiation with 6 Gy X-rays (D). Scale bars, 500 μm; (E) and (F) are quantifications of the relative sizes of spheroids in the experiments shown in (C) and (D), respectively.

FIGS. 5 G and H: Invasion of H4 spheroids after 24 h and 72 h of incubation with 25 μM of FeL and CuL and 12.5 μM and 25 μM of the copper complex obtained according to example 6 (“CuL₂”).

FIG. 6: Reduction of U87-MG cell line invasion of Matrigel™ by compounds of the invention in a spheroid invasion model; reduction in % compared to DMSO control was at 24H: 6.9% for FeL and 39.1% for CuL, and for 48H: 16.3% for FeL and 44.0% for CuL (average of three independent assays using 2-4 spheroids per condition per assay in each case).

FIG. 7: Reduction of U87-MG cell line invasion of Matrigel™ by compounds of the invention in a spheroid invasion model; reduction in % compared to control for the copper complex obtained according to example 6 (“CuL₂”) and CuL (average of three independent assays using 2-3 spheroids per condition per assay in each case).

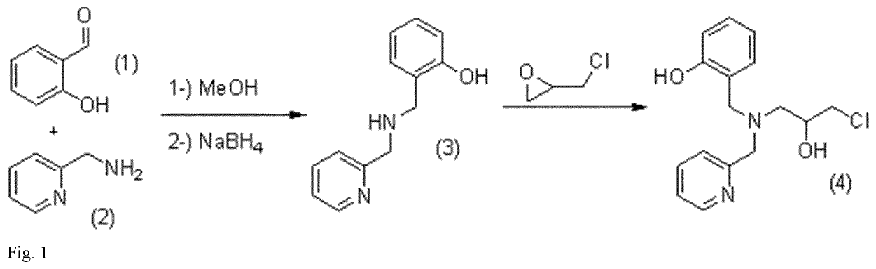

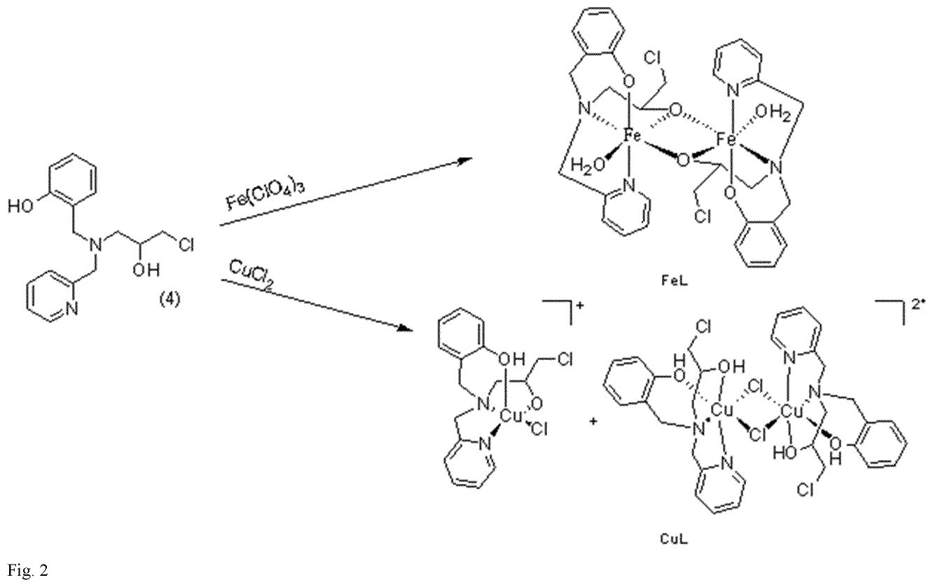

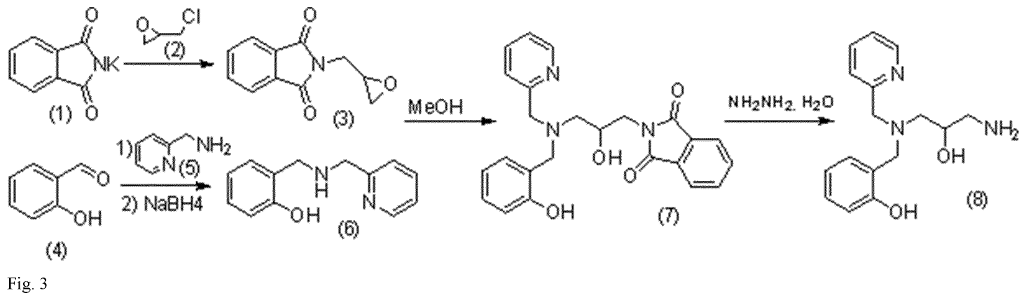

The following Examples shall merely illustrate the invention. They shall not be construed, whatsoever, to limit the scope of the invention.

### EXAMPLE 1: SYNTHESIS OF COMPLEX CUL AND FEL

Synthesis of compounds FeL and CuL described previously on the following publications: Horn, A. et al. Synthesis, crystal structure and properties of dinuclear iron(III) complexes containing terminally coordinated phenolate H2O/OH-groups as models for purple acid phosphatases: efficient hydrolytic DNA cleavage. Inorganica Chim. Acta 358, 339-351 (2005); Horn Jr., A. et al. Synthesis, molecular structure and spectroscopic, electrochemical and magnetic properties of a new dinuclear iron complex containing, μ-sulfate-di-μ-alkoxo bridges: evaluating the influence of the sulfate bridge on the physicochemical properties of the di-μ-alkoxo-diiron unit. J. Braz. Chem. Soc. 17, 1584-1593 (2006); and Fernandes, C. et al. Synthesis, characterization and antibacterial activity of FeIII, CoII, CuII and ZnII complexes probed by transmission electron microscopy. J. Inorg. Biochem. 104, 1214-1223 (2010).

### EXAMPLE 2: SYNTHESIS OF 2-OXIRANYLMETHYL-ISOINDOLE-1,3-DIONE (3)

16.0 g (86.7 mmol) of compound (2) in 45 mL (573.9 mmol) of epichlorohydrin (3) were placed in a 125 mL flask. The reaction was refluxed for 24 hours and then the excess of compound 3 was removed by vacuum distillation. A white solid was obtained which was then treated with hot methanol and filtered while still hot. The solution obtained was brought to the refrigerator for 24 hours to obtain 11.0 g (mmol) of the product (4). White solid; yield of 63%; ¹H RMN (CDCl₃, 500 MHz/ppm): δ 7.88 (dd, J=5.5 Hz, 3.0 Hz, 2H, CH_{arom.}), 7.83 (dd, J=5.5, 3.0 Hz, 2H, CH_{arom.}), 3.89 (dd, J=14.53, 5.1 Hz, 1H, CH₂), 3.83 (dd, J=14.53, 5.1 Hz, 1H, CH₂), 3.26-3.21 (m, 1H, CH), 2.81 (t, J=4.6 Hz, 1H, CH₂), 2.65 (dd, J=4.6, 2.6 Hz, 1H, CH₂). ¹³C RMN (CDCl₃, 125 MHz/ppm): δ 167.9 (2×C), 134.1 (2×CH), 131.9 (2×C), 123.4 (2×CH), 49.0 (CH), 46.1 (CH₂), 39.6 (CH₂).

### EXAMPLE 3: SYNTHESIS OF 2-{[(PYRIDIN-2-YLMETHYL)-AMINO]-METHYL}-PHENOL (6)

4.35 mL (41.0 mmol) of 2-hidroxibenzaldehyde and 4.22 mL (41.0 mmol) of 2-aminemethilpyridine were added in a 125 mL flask in 50 mL of methanol. The reaction was stirred for 30 min. at room temperature. Then, 1.52 g (41.0 mmol) of Sodium borohydride were added slowly in an ice bath and the reaction was stirred for 24 hours more. The reaction was concentrated and extracted with dichloromethane x brine. The organic phase was treated with sodium sulfate anhydrous, providing an orange oil, and left in a beaker for crystallization. The obtained solid was macerated, washed with cold isopropanol, vacuum filtered and dried in the desiccator. Obtained 7.00 g (32.6 mmol) of white solid with yield of 80%. ¹H RMN (CDCl₃, 500 MHz/ppm): δ 8.58 (s, 1H, CH_{arom.}), 7.66 (d, J=7.6, 1.7 Hz, 1H, CH_{arom.}), 7.23-7.18 (m, 3H, CH_{arom.}), 6.97 (d, J=7.30 Hz, 1H, CH_{arom.}), 6.86 (dd, J=8.1, 0.6 Hz, 1H, CH_{arom.}), 6.78 (dt, J=7.3, 0.6 Hz, 1H, CH_{arom.}), 4.01 (s, 2H, CH₂), 3.93 (s, 2H, CH₂). ¹³C RMN (CDCl₃, 125 MHz/ppm): δ 158.2 (C), 157.8 (C), 149.5 (CH), 136.7 (CH), 128.8 (CH), 128.6 (CH), 122.7 (C), 122.5 (CH), 119.1 (CH), 116.5 (2×CH), 53.1 (CH₂), 51.9 (CH₂).

### EXAMPLE 4: SYNTHESIS OF 2-{2-HYDROXY-3-[(2-HYDROXY-BENZYL)-PYRIDIN-2-YLMETHYL-AMINO]-PROPYL}-ISOINDOLE-1,3-DIONE (7)

5.54 g (25.8 mmol) of product 6 and 5.24 g (25.8 mmol) of product 3 were added in 50 mL of methanol in a 125 mL Ambar flask and the reaction was stirred at room temperature for 96 hours. The solid precipitated and was vacuum filtered, washed with cold isopropanol and dried in the desiccator. Obtained 7.55 g (18.00 mmol) of white solid with yield of 70%. ¹H RMN (CDCl₃, 500 MHz/ppm): δ 8.53 (ddd, J=4.88, 2.59, 0.76 Hz, 1H, CH_{arom.}), 7.81-7.77 (m, 2H, CH_{arom.}), 7.77-7.70 (m, 2H, CH_{arom.}), 7.62 (m, 1H, CH_{arom.}), 7.19-7.16 (m, 1H, CH_{arom.}), 7.13-7.07 (m, 2H, CH_{arom.}), 6.97 (dd, J=7.47, 1.52 Hz, 1H, CH_{arom.}), 6.75 (dd, J=8.09, 1.07 Hz, 1H, CH_{arom.}), 6.71 (dt, J=7.47, 1.21 Hz, 1H, CH_{arom.}), 4.20-4.14 (m, 1H, CH), 4.02 (d, J=15.4, 1H, CH₂), 3.93 (d, J13.4, 1H, CH₂), 3.86 (d, J15.4, 1H, CH₂), 3.72-3.68 (m, 2H, CH₂), 2.75-2.71 (m, 2H, CH₂), 3.65-3.60 (m, 1H, CH₂). ¹³C RMN (CDCl₃, 125 MHz/ppm): δ 181.5 (2×C), 181.2 (2×C), 161.0 (C), 156.0 (C), 148.9 (CH), 137.3 (CH), 134.1 (2×CH), 129.3 (2×CH), 123.4 (3×CH), 122.7 (C), 122.6 (CH), 119.2 (CH), 116.7 (CH), 67.2 (CH), 58.5 (2×CH₂), 58.0 (CH₂), 42.2 (CH₂).

### EXAMPLE 5: SYNTHESIS OF 2-{[(3-AMINO-2-HYDROXY-PROPYL)-PYRIDIN-2-YLMETHYL-AMINO]-METHYL}-PHENOL (8)

7.55 g (18.0 mmol) of product 7 and 1.81 g (36.00 mmol) of hydrazine monohydrate were added in 50 mL of ethanol in a 125 mL flask, and the reaction was refluxed for 10 minutes. When a white insoluble product was observed, the solution was acidified until pH 4.0 with HCl _{(conc.)} and vacuum filtered. Then the solution was concentrated, basified with NaOH 5M until pH 10.0 and successive extractions with DCM were done. The organic phase was treated with sodium sulfate sodium sulfate anhydrous, providing 3.37 g of an orange oil with yield of 65%. IR (KBr, cm⁻¹): 3479, 3410, 3321, 3209, 2829, 1676, 1653, 1489, 1411, 758. ¹H RMN (CDCl₃, 500 MHz/ppm): δ 8.57 (d, J=4.28 Hz, 1H, CH_{arom.}), 7.64 (dt, J=7.7, 1.1 Hz, 1H, CH_{arom.}), 7.22-7.13 (m, 3H, CH_{arom.}), 6.99 (dd, J=7.5, 1.1 Hz, 1H, CH_{arom.}), 6.83 (dd, J=7.7, 1.1 Hz, 1H, CH_{arom.}), 6.76 (dt, J=7.7, 1.1 Hz, 1H, CH_{arom.}), 4.00 (d, J=15.3 Hz, 1H, CH₂), 3.89 (m, 1H, CH₂), 3.85 (d, J=15.3 Hz, 1H, CH₂), 3.83-3.77 (m, 1H, CH), 3.69 (d, J=13.6 Hz, 1H, CH₂), 2.74-2.56 (m, 4H, CH₂). ¹³C RMN (CDCl₃, 125 MHz/ppm): δ 157.6 (C), 157.5 (C), 150.0 (CH), 137.4 (CH), 129.7 (CH), 129.2 (CH), 123.4 (CH), 122.6 (C), 122.6 (CH), 119.2 (CH), 116.7 (CH), 69.5 (CH), 59.0 (CH₂), 58.4 (CH₂), 57.9 (CH₂), 45.6 (CH₂).

### EXAMPLE 6: SYNTHESIS OF COMPLEX BETWEEN LIGAND 8 AND CuCL₂ (“CuL₂”)

0.287 g (1.00 mmol) of compound 8 in 10 mL of isopropanol were stirred in reflux for 10 minutes, then 0.170 g (1.00 mmol) of CuCl₂ were added. H₂O solubilized in 10 mL of isopropanol was added in the reaction and refluxed for 2 hours. The reaction medium was cooled to room temperature and the precipitate formed was vacuum filtered. Elemental Analysis (% CHN) found: C 39.49 H 5.31 N 7.85. Mass ESI+(m/z): 554.0997; 697.1631; 796.0520. The complex is water soluble, such as in a concentration of 1 mM.

### EXAMPLE 7: CELL CULTURE

Human brain neuroglioma (H4) cells (ATCC, Manassas, Va., USA) were grown in Dulbecco's Modified Eagle's Medium (DMEM) supplemented with 10% Fetal Bovine Serum (FBS) and 1% Penicillin/Streptomycin. Human brain glioblastoma (U-87 MG) cells were grown in Minimum Essential Medium (MEM) supplemented with 10% Fetal Bovine Serum (FBS) and 1% Penicillin/Streptomycin. All medium and supplements were obtained from Gibco™ Invitrogen. The cell lines were cultured continuously as a monolayer at 37° C. and 5% of C02 for no more than 20 passages at a time after resuscitation.

### EXAMPLE 8: CELL CYCLE ASSAY

For the cell cycle assay, 7×105 cells were seeded in 75 cm² flasks and incubated for 24 hours at 37° C. The medium was removed and cells were washed once with DPBS before 10.5 mL of fresh medium, solutions of 24, 16 and 8 μM of FeL and CuL, or a solution of 0.125% of DMSO (the concentration relative to the highest compound's concentration used—24 μM) were added to the flasks. The flasks were incubated for an additional 24 hours, after which cells were detached, washed with PBS, and 10⁶ cells were fixed through drop by drop addition of 70% cold ethanol (v/v in DPBS) under gently vortexing. Samples were stored at 4° C. for 24 hours, centrifuged and the supernatant was removed. Subsequently, 250 μL of RNase A (10 mg/mL in PBS; Sigma Aldrich, St. Louis, Mo., USA) were added to each sample, which was then incubated at room temperature for 30 min and washed twice with DPBS. In the dark, each sample was stained with 20 μg/mL of propidium iodide (PI) (eBioscience, San Diego, Calif., USA) for 15 min before being analyzed using a flow cytometer (BD FACS CANTO™ II).

### EXAMPLE 9: TRANSWELL MIGRATION ASSAY

Cells starved overnight were detached and seeded onto cell culture inserts in 24-well plates (Millipore transwell PET filters, 8 μm pore; Merck, Kenilworth, N.J., USA) at a density of 1.0×10⁴ cells in 150 μL of FBS-free medium, or FBS-free medium containing 0.125% DMSO, 25 μM of FeL or 25 μM of CuL. The lower transwell chambers were filled with 600 μL of media without FBS (negative control) or with medium containing 10% FBS. After 24 h of incubation at 37° C., the inserts were washed with DPBS, fixed with 4% paraformaldehyde, washed again, and stained with 1 g/mL of Hoechst 33342 (Thermo Scientific, Waltham, Mass., USA) for 20 min at room temperature. Cells were then imaged at a 200× amplification on a confocal microscope (Zeiss LSM 710). Seven random fields were photographed per insert, with at least two inserts being analyzed for each condition per experiment. The results shown were calculated based on three independent experiments.

### EXAMPLE 10: SPHEROIDS VIABILITY ASSAY

For spheroids formation, 2.5×10³ cells were seeded in 100 μL/well in 96-well plates coated with 1.5% agarose (w/v in PBS). After 1 day of incubation, spheroids were fully formed, and 100 μL of fresh medium or medium with DMSO or the compounds was added to a final concentration of 0.125% and 25 μM, respectively. Cells were incubated for 24 h or 72 h at 37° C. before cell viability was estimated using the CellTiter-Glo® 3D assay (Promega, Madison, Wis., USA) according to the manufacturer's instructions. Luminescence was read in a CLARIOstar® microplate reader (BMG LABTECH). In addition, spheroids' viability was also estimated based on spheroids' growth. For that, the total area of each spheroid was determined using the INSIDIA macro in FIJI, and then normalized to the area of the spheroid at day 0 (to account for possible differences in the spheroids initial size) and to the size of the untreated spheroids at each time point (to assess the effect of the DMSO and the compounds on spheroid growth).

### EXAMPLE 11: SPHEROID INVASION ASSAY

Each one-day old spheroid, formed as described above, was collected into a tube, washed once with FBS-free medium, and resuspended in 40 μl of a 4.5 mg/ml Matrigel™ (Corning #356231) solution in FBS-free medium. Then, each spheroid-containing suspension was spotted onto the centre of a well of a 24-wells plate and incubated as a hanging drop for 1 h until the matrigel had polymerized. Complete medium, complete medium with 0.125% DMSO, or complete medium containing 24 μM of the compounds were added and the spheroids were incubated for 24 h at 37° C. before being irradiated (or not as a control) with 6 Gy X-rays on a Faxitron MultiRad225. Images of spheroids and invading cells were acquired immediately after embedment and every 24 h after that, using an Eclipse Ts2 microscope (Nikon). At each time point (24 h, 48 h, and 72 h) the total area of the spheroid and invading cells was determined as described above.

### EXAMPLE 12: FEL AND CUL COMPLEXES INHIBIT MIGRATION THROUGH INDUCTION OF MESENCHYMAL-EPITHELIAL TRANSITION (MET) IN GLIOMA CELLS

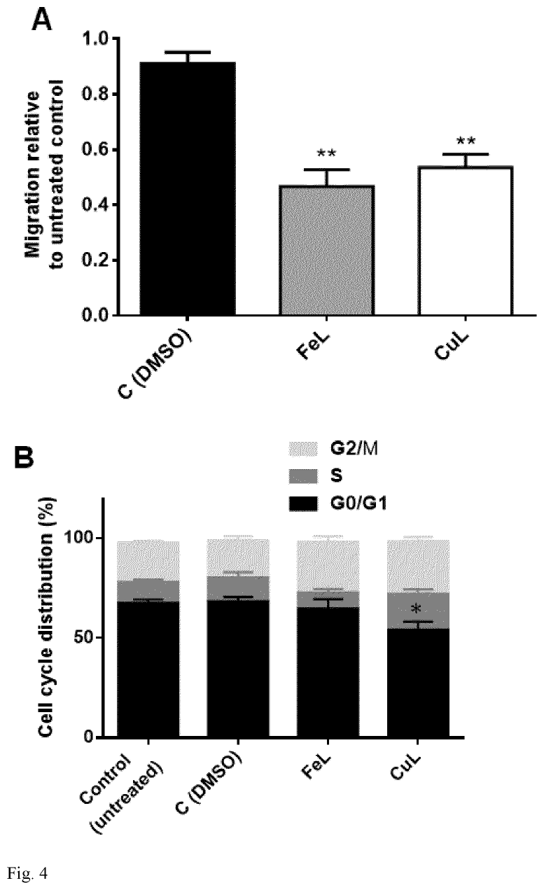

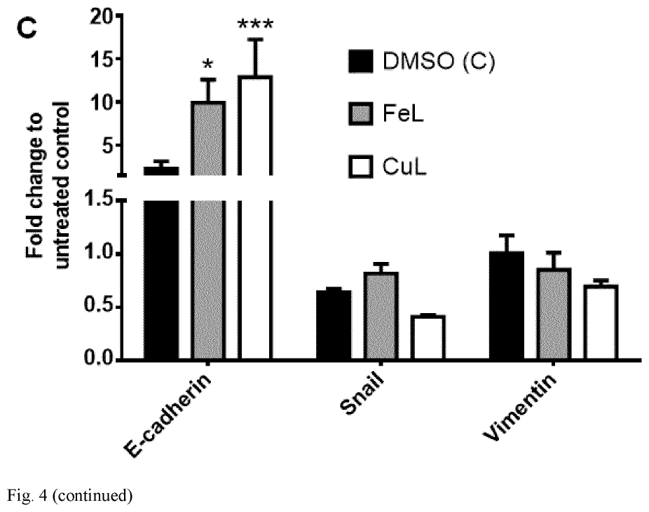

The effect of FeL and CuL on the migration of H4 cells was thus investigated by the transwell assay. The number of cells migrated to the bottom of the membrane revealed that both compounds can clearly inhibit the migratory ability of H4 cells (FIG. 4A). To investigate to what extent this observation was related to cell proliferation or cell cycle arrest induction, the effects of the compounds on the cell cycle of H4 cells were investigated by flow cytometry. While FeL showed no effect on the cell cycle of H4 cells, CuL induced a significant decrease in the G0/G1 phase of the cycle (*P<0.05), with a concomitant increase in the % of cells in the S and G2/M phases (of about 7.7 and 6.2%, respectively) that was, however, statistically not significant (FIG. 4B). Regardless, despite the fact that exit from the S phase of the cycle, as well as entry or exit of the G2/M was slightly affected in response to CuL, this difference does not justify the significant difference observed in cells' migration upon exposure to the compound. As such, looking for another possible explanation, we next analyzed the expression of several EMT markers in the FeL/CuL treated cells by qPCR. The results evidenced that treatment with the compounds is accompanied by a statistically significant increase in expression of E-cadherin, and a slight reduction of vimentin (FIG. 4C). The expression of the EMT-related transcription factor snail was found to also be decreased upon treatment with CuL. This expression profile is consistent with the hypothesis that cells treated with FeL and CuL experienced a MET transition, which should originate cells with a less motile phenotype, and is in accordance with the decreased migratory ability observed in compound-treated cells, demonstrating that the compounds do possess anti-metastatic properties.

### EXAMPLE 13: COMPLEXES OF THE PRESENT INVENTION INHIBIT 3D SPHEROIDS INVASION

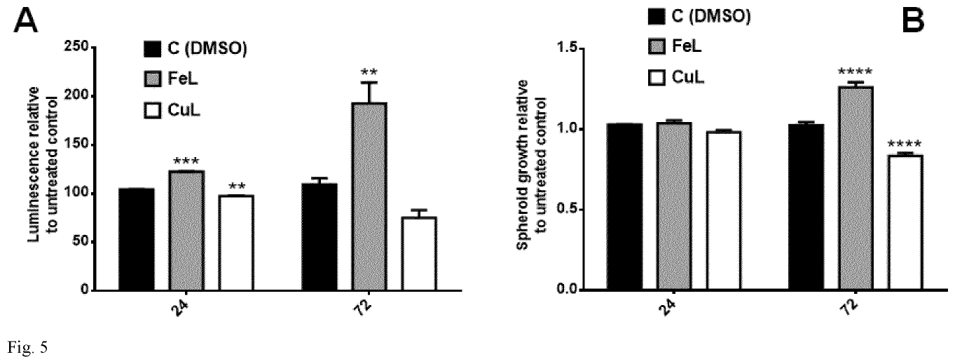

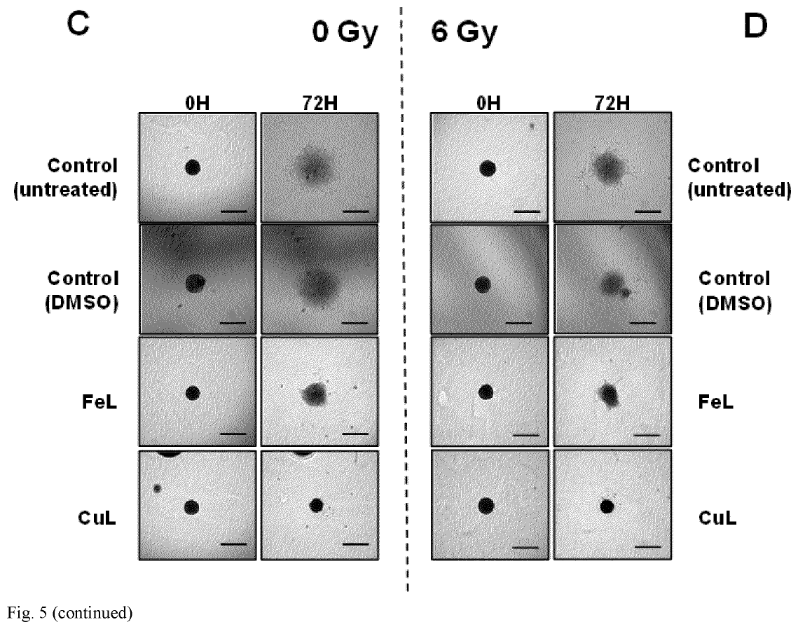

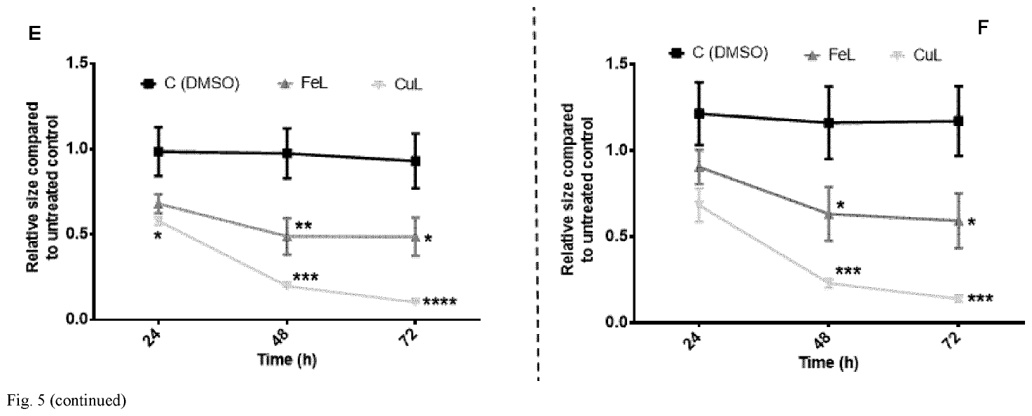

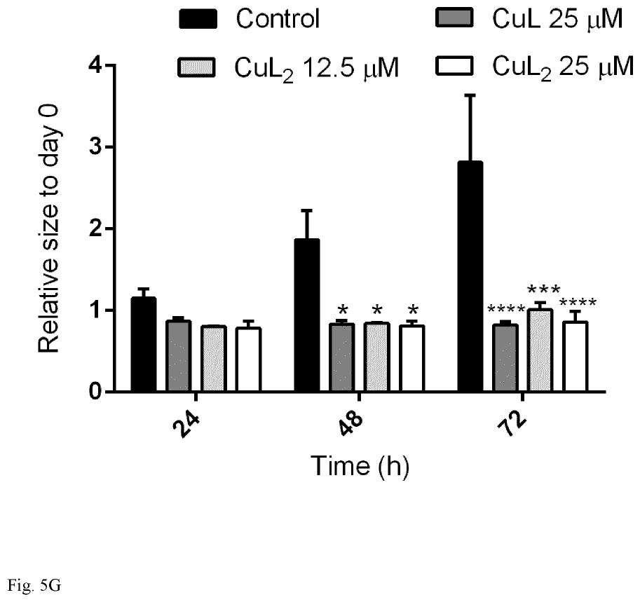

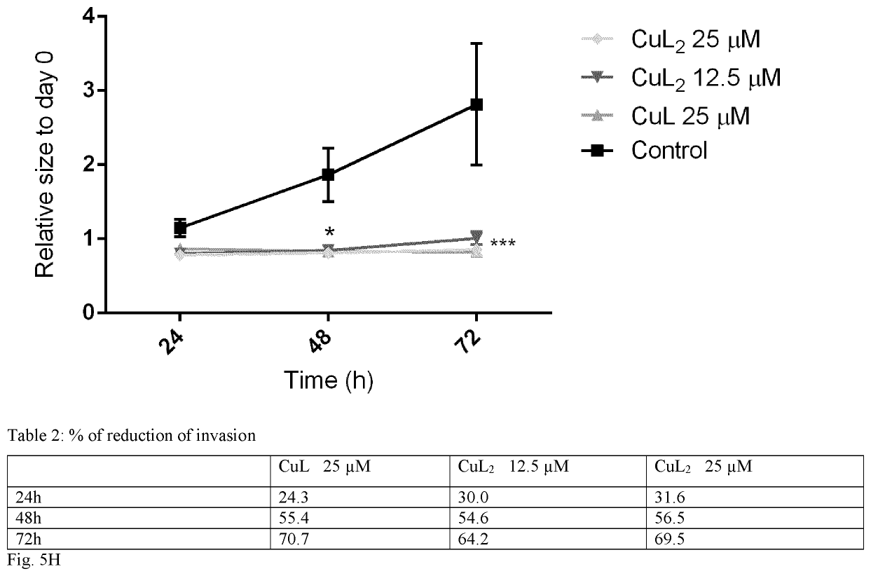

Several 3D cellular models have been developed that present a level of complexity which is much closer and more representative of several aspects of tumor tissues than the ones shown by monolayer cell cultures. In particular, matrix-embedded 3D cultures have been more and more applied to investigate tumor migration and invasion. As such, in order to try to better estimate the clinical translational potential of the compounds under study, we extended our studies to H4 multicellular spheroids, which are expected to better recapitulate in vivo tumor properties. For that purpose, spheroids generated in agarose-coated plates were first treated with FeL or CuL for up to 24 h or 72 h. Then, cell viability was assessed using the CellTiter-Glo® 3D assay, while spheroid size and growth was accompanied using classical bright field microscope. Surprisingly, incubation with FeL increased cellular viability (FIG. 5A), both after 24 and 72 h of incubation. This increase in viability was accompanied by an increase in spheroid size after 72 h of incubation (FIG. 5B). In contrast, CuL induced a decrease in viability as early as after 24 h of incubation, along with a concomitant decrease in spheroid size (FIGS. 5A and 5B).

Next, we observed that the compounds FeL and CuL and CuL₂ were able to interfere with the invasive behavior exhibited by H4 cells embedded in matrigel, both without and after irradiation with 6 Gy X-rays (FIGS. 5C, E and 5D, F, G and H respectively). CuL and CuL₂ in particular completely eliminated H4 cells' ability to invade the matrigel matrix, an effect that, in the case of CuL, cannot be attributed solely to the slight decrease in viability found to occur following incubation with this compound. FeL also displayed an ability to inhibit the invasive behavior of H4 cell. The results obtained in the 3D invasion assays thus clearly demonstrate that the compounds possess an anti-metastatic effect not only in monolayer cells, but also in the more representative spheroids model.

### EXAMPLE 14: REDUCTION OF U87-MG CELL LINE INVASION OF MATRIGEL™

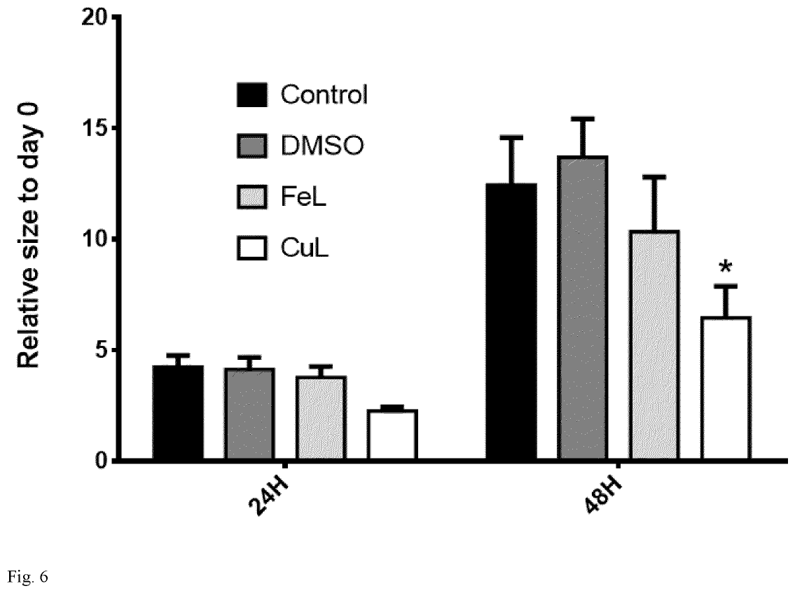

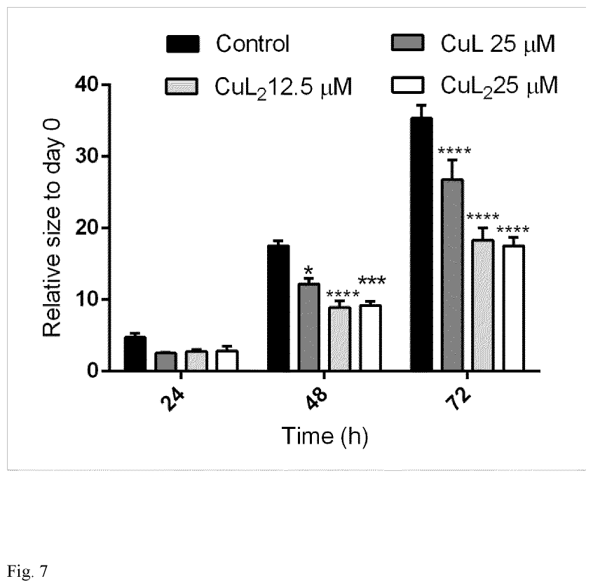

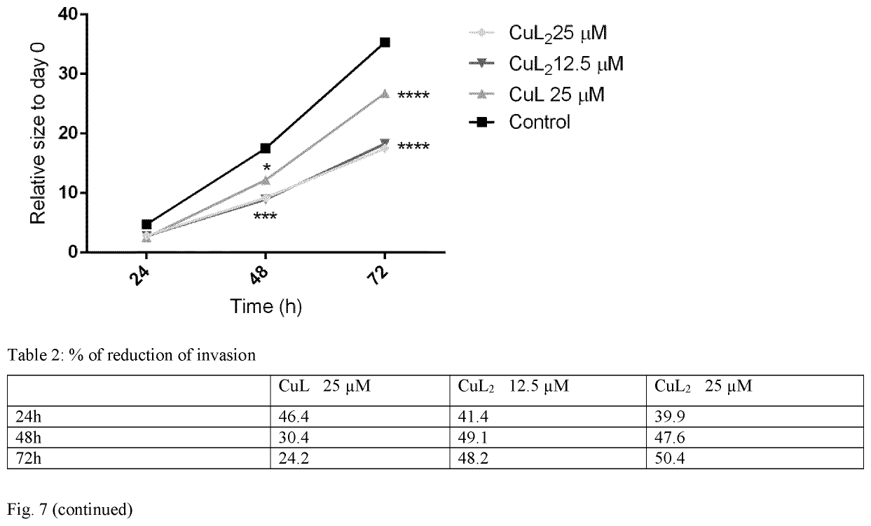

The effect of the compounds of the invention on the invasion into Matrigel™ by the highly invasive glioma cell line U87-MG cell line was determined in a spheroid invasion model in analogy to the above. For spheroids formation, 2.0×10³ cells were seeded in 100 μL/well in Nunclon™ Sphera™ Microplates. After 3 days of incubation, spheroids were fully formed and were embedded in 4.5 mg/ml Matrigel™ as described above. Invasion was assessed at 24 h, 48 h and 72 h after embedment, in the absence of irradiation. As shown in FIG. 6 and FIG. 7, invasion by this cell line is also inhibited by the compounds of the invention.

### LITERATURE CITED

BR 10 2014 022630 A2
BR 10 2014 017397 A2
Chen, et al. (2017), J. Pharmacol. Sci. 134, 59-67
Fernandes et al. (2010) J. Inorg. Biochem. 104, 1214-1223
Gu et al. (2019), Eur. J. Med. Chem. 164, 654-664
He et al. (2017), J. Organomet. Chem. 842, 82-92
Horn et al. (2005) Inorganica Chim. Acta 358, 339-351
Horn Jr. et al. (2006) J. Braz. Chem. Soc. 17, 1584-1593

1-22. (canceled)

23. A method for preventing migration of cancer cells in a subject known or suspected to suffer from cancer with a composition comprising at least one metal complex having the structure (I): 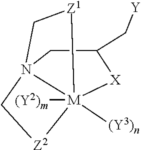
wherein:
M is a metal, preferably selected from the group consisting of copper, iron, manganese and zinc;
X is X^{a} or X^{b}, wherein X^{a} is selected from the group consisting of O, S and —N(R¹)—, wherein R¹ is H or alkyl, and wherein X^{b} is a group forming a coordinate covalent bond to a second metal M′, preferably a group O, S or —N(R¹)—, wherein M′ is preferably selected from the group consisting of copper, iron, manganese and zinc, and wherein M′ and M may be the same or different and are preferably the same;
Z¹ and Z², are independently of each other a, substituted or unsubstituted, -Aryl-O—, -Aryl-N— or heteroaryl group;
Y is Y^{a} or Y^{b}, wherein Y^{a} is selected from the group consisting of H, alkyl, —OH, —SH, halogen, and —NR³R⁴, wherein R³ and R⁴, are independently of each other selected from H and alkyl, preferably R³ and R⁴ are both H, and wherein Y^{b} is a group forming a coordinate covalent bond to M or M′, preferably Y^{b} is a group O, S or —N(R^{Yb1}R^{Yb2}) wherein R^{Yb1} and R^{Yb1}, are, independently of each other, H or alkyl, preferably H;
n and m are integers, which are independently of each other, 0 or 1;
Y² is water or a halogen; and
Y³ is water or a halogen.

24. The method of claim 23, wherein the complex is a mononuclear or binuclear complex or a mixture thereof.

25. The method of claim 23, wherein Y is Y^{a} and wherein the complex has one of the following structures: 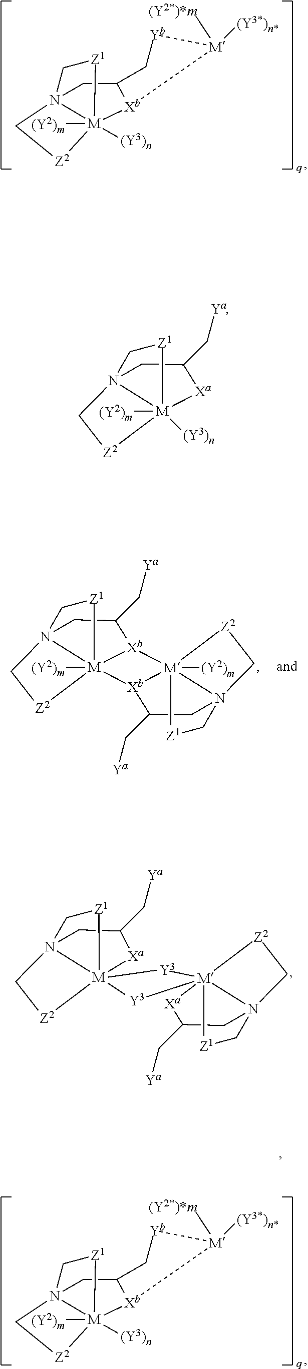
preferably
wherein:
M and M′ are metals, preferably selected from the group consisting of copper, iron, manganese and zinc, wherein M′ and M may be the same or different and are preferably the same;
X^{b} is a group O, S or —N(R¹)—, wherein R¹ is H or alkyl;
Z¹ and Z², are independently of each other a, substituted or unsubstituted, -Aryl-O—, -Aryl-N— or heteroaryl group;
Y^{b} is a group O, S or —N(R^{Yb1}R^{Yb2}), wherein R^{Yb1} and R^{Yb1} are, independently of each other, H or alkyl, preferably H;
Y² is water or a halogen;
y^{2*} is a solvent molecule or a halogen, preferably water, methanol or a halogen, more preferably, water, methanol or —Cl;
Y³ is water or a halogen;
Y^{3*} is a solvent molecule or a halogen, preferably water, methanol or a halogen, more preferably, water, methanol or —Cl;
n and m are integers, which are independently of each other, 0 or 1;
n* and m* are integers, which are independently of each other, 0 or 1; and
q is an integer of from 2 to 5, preferably q is 2 or 3, more preferably 2.

26. The method of claim 23, wherein Y is Y^{b} and wherein the complex has the structure:

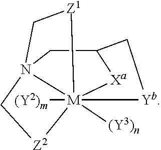

27. The method of claim 23, wherein M and M′ are, independently of each other selected from the group consisting of Fe(III), Cu(II), Mn(II) and Zn(II), with Fe(III) and Cu(II) being particularly preferred.

28. The method of claim 23, wherein the complex has a structure selected from the group consisting of:

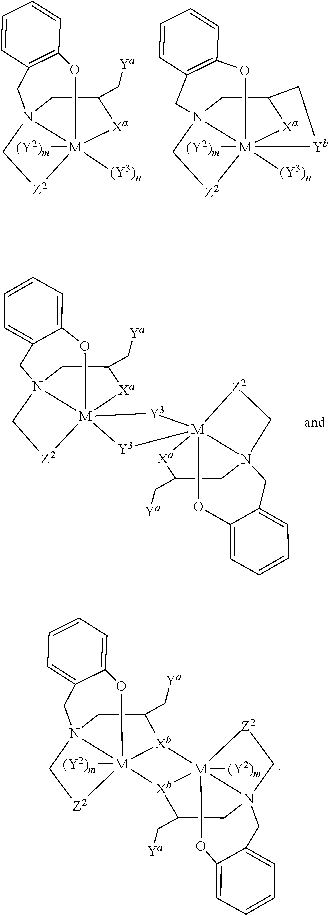

29. The method of claim 23, wherein the complex has a structure selected from the group consisting of:

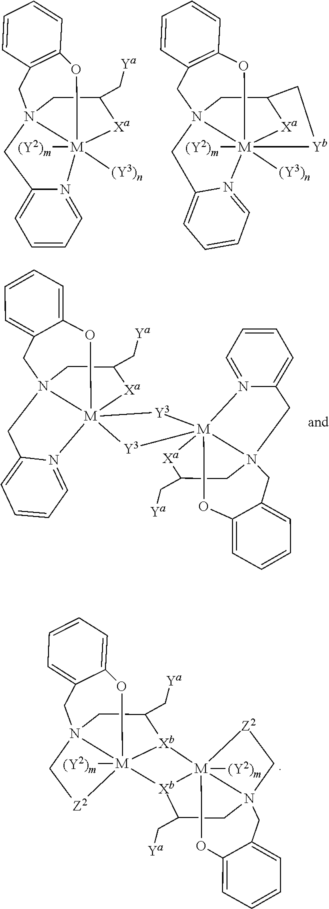

30. The method of claim 23, wherein the complex has as structure selected from the group consisting of: 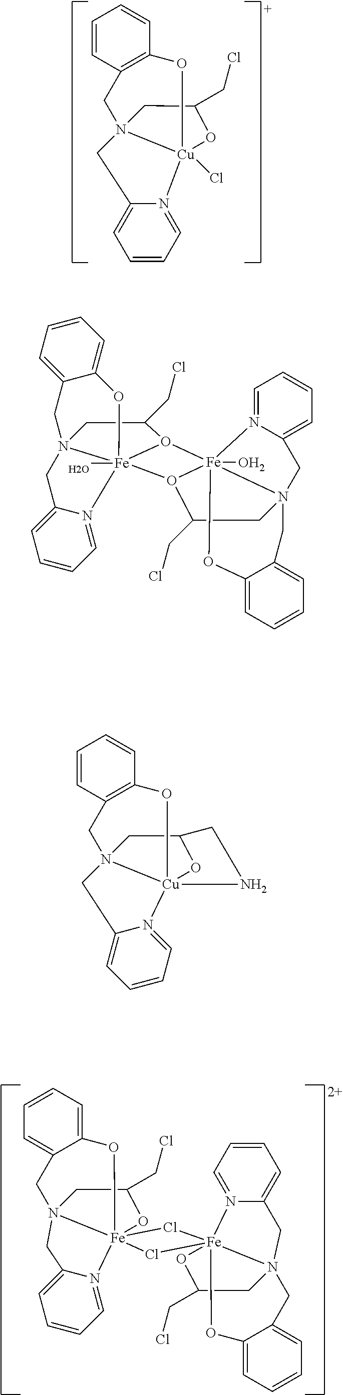
and mixtures of two or more thereof.

31. A method for preventing migration of cancer cells in a subject known or suspected to suffer from cancer with a composition comprising a metal complex, wherein the metal complex is obtained or obtainable by reacting a metal salt comprising a metal ion M* with a ligand having the structure (I*): 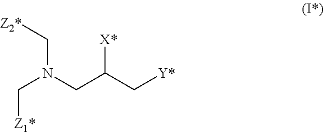
wherein:
X* is selected from the group consisting of —OH, SH, and —N(R¹)(R²)—, wherein R¹ is H or alkyl, and wherein R² is H or alkyl;
Z^{1*} and Z^{2*} are independently of each other, a substituted aryl or a, substituted or unsubstituted, heteroaryl group; wherein the substituted aryl group is preferably substituted with an hydroxyl or amine group;
Y* is selected from the group consisting of H, alkyl, —OH, —SH, halogen and —NR³R⁴; and
wherein M* is selected from the group consisting of copper, iron, and manganese.

32. The method of claim 31, wherein the ligand is selected from the group consisting of.

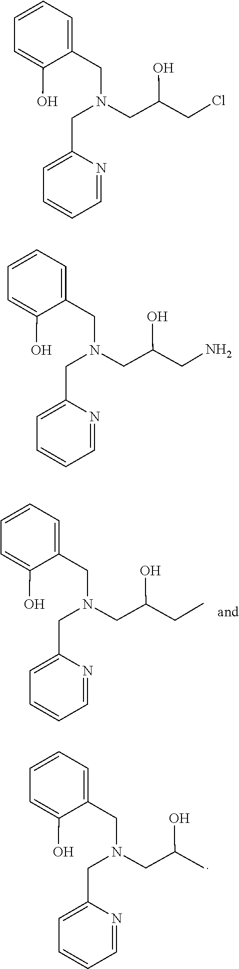

33. The method of claim 23, wherein the preventing migration of cancer cells is preventing metastasis, tissue invasion, and/or relapse.

34. The method of claim 23, wherein the composition is administered before tumor removal and/or wherein the composition is administered at a site of tumor removal.

35. The method of claim 23, wherein the cancer is brain cancer, colorectal cancer, breast cancer, pancreatic cancer, lung cancer, bladder cancer, prostate cancer, or ovarian cancer, preferably is brain cancer, more preferably glioma.

36. A method of claim 23, wherein the composition is combined with a cancer therapeutic agent.

37. A method, preferably an in vitro method, for determining whether cancer cells are susceptible to immobilizing by a composition as specified in claim 23, comprising contacting the cancer cells with the composition and determining cancer cells to be susceptible to immobilizing by the compound in case the cancer cells are found to be immobilized.

38. The method of claim 23, comprising contacting cancer cells with the compound as specified in claim 23, and thereby preventing migration of cancer cells.

39. A method for treating cancer, comprising the steps of the method for preventing migration of cancer cells according to claim 38, and administering at least one anticancer therapy.

40. The method of claim 39, wherein the anticancer therapy is selected from the list consisting of (i) radiotherapy, (ii) chemotherapy, (iii) anti-hormone therapy, (iv) targeted therapy, (v) immunotherapy, and (vi) any combination of (i) to (v).

41. A complex having the structure: 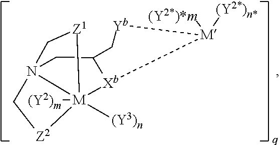
wherein:
M and M′ are metals, preferably selected from the group consisting of copper, iron, manganese and zinc, wherein M′ and M may be the same or different and are preferably the same;
X^{b} is a group O, S or —N(R¹)—, wherein R¹ is H or alkyl;
Z¹ and Z², are independently of each other a, substituted or unsubstituted, -Aryl-O—, -Aryl-N— or heteroaryl group;
Y^{b} is a group O, S or —N(R^{Yb1}R^{Yb2}), wherein R^{Yb1} and R^{Yb1} are, independently of each other, H or alkyl, preferably H;
Y² is water or a halogen;
Y^{2*} is a solvent molecule or a halogen, preferably water, methanol or a halogen, more preferably, water, methanol or —Cl;
Y³ is water or a halogen;
Y^{3*} is a solvent molecule or a halogen, preferably water, methanol or a halogen, more preferably, water, methanol or —Cl;
n and m are integers, which are independently of each other, 0 or 1;
n* and m* are integers, which are independently of each other, 0 or 1; and
q is an integer of from 2 to 5, preferably q is 2 or 3, more preferably 2.

42. The complex of claim 41, having the structure: 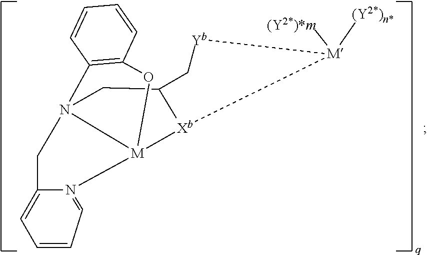
preferably wherein M and M′ are copper, Y^{b} is NH₂, X^{b} is 0, m* and n* are both 1, wherein Y²* and Y³* are Cl, and q is an integer in the range of from 2 to 5, preferably 2 or 3, more preferably 2.
